(19) United States
(12) Patent Application Publication
(10) Pub. No.: US 2023/0250385 A1
(43) Pub. Date: Aug. 10, 2023

(54) AN INCUBATOR FOR RECEIVING A NUMBER OF CELL CULTURE CHAMBER DEVICES

(71) Applicant: CelVivo ApS, Blommenslyst (DK)

(72) Inventors: Hans Henrik Jochumsen, Blommenslyst (DK); Krzysztof Wrzesinski, Blommenslyst (DK); Stephen John Fey, Blommenslyst (DK)

(21) Appl. No.: 18/015,152

(22) PCT Filed: Jun. 29, 2021
(86) PCT No.: PCT/EP2021/067777

§ 371 Date: Jan. 9, 2023

(30) Foreign Application Priority Data
Jul. 10, 2020 (DK) PA 2020 70482

(51) Int. Cl. C12M 1/00; C12M 1/36; C12M 3/04; C12M 1/12

(52) U.S. Cl. CPC C12M 41/14; C12M 41/48; C12M 41/06; C12M 23/22; C12M 27/10; C12M 25/06

(57) ABSTRACT

An incubator configured to receive a predetermined number of cell culture chamber devices, each cell culture chamber device including an enclosure configured to contain a cell culture media. The incubator has a housing including an incubation chamber configured to contain at least a respective part of the cell culture chamber devices when received by the incubator, and at least one registration and/or detection device being integrated with the incubator and being configured to register and/or detect an illumination or visualisation signal after passing, reflecting, or propagating through at least a part of the enclosure of at least one of the predetermined number of cell culture chamber devices when received by the incubator.

### FIELD OF THE INVENTION

The present invention relates generally to an incubator configured to receive a predetermined number of cell culture chamber devices (also equally referred to herein as bioreactors) where each cell culture chamber device comprises an enclosure (also equally referred to herein as a cell chamber) configured to contain a cell culture media (typically comprising cells). Additionally, the present invention relates to an incubator system comprising a first incubator and at least a second incubator.

### BACKGROUND

When growing cells and tissue using more traditional cell culture chamber devices, often having an essentially flat cell support surface or the like, primary cells and biopsies tend to de-differentiate and lose their normal structural organisation and in vivo functionality. One example of this is where cells migrate from a block of tissue out onto the flat supporting surface (i.e. the so-called “melting ice-cream effect”). De-differentiated cells typically express different biochemical properties than those normally expressed by corresponding cells in an intact organism. Furthermore, certain cells have typically lost their specialised functions compared to corresponding cells in an intact organism.

Improving on this, certain cell culture chamber devices or bioreactors for the growing of cell cultures, whether a single or several cell types, or tissues, normally or even preferably use operation under omnidirectional normogravity conditions i.e. clinostat induced conditions, since this enables the preservation of the differentiated state of many types of cell in the culture. Furthermore it promotes the recovery, (or re-differentiation) of in vivo like structure and functionality in many different cell lines. This is significant because cell lines are used for the majority of cell culture work currently executed.

Such omnidirectional normogravity conditions may be induced by continuous rotation of the compartment containing the cell culture, thereby preventing the cells to adhere to the compartment walls (strictly speaking, the rotation infinitesimally increases the gravitational force (centripetal acceleration)). Suitable rotation promotes the adherence of cells to each other in a fluid environment with a minimum of shear forces acting on the culture. Shear forces can be introduced, if needed, for specific cell/tissue types, by changing the rotation speed of the bioreactor. Thereby cells aggregate into colonies typically named spheroids or organoids (in this disclosure referred to collectively as spheroids). Since pieces of tissue will be affected similarly, they are also included under the generic term spheroids.

As the spheroids grow, they get bigger and thus the rate of rotation of the bioreactor needs, for certain uses, to be adjusted to maintain optimal conditions where the spheroids remain in an essentially ‘stationary orbit’ relative to the bioreactor as this promotes improved uniformity of the spheroids. For other uses, the spheroids should not or need not remain in a stationary orbit but rather be allowed a different behaviour e.g. be allowed to tumble or be located on or near the bottom of the cell culture chamber, or be held against the wall of the cell culture chamber by centripetal acceleration, etc. However, in any event it is very beneficial to be able to clearly inspect the spheroids in the bioreactor at several occasions e.g. to see whether a speed adjustment should be made, and potentially to what extent.

Improved uniformity of the spheroids results in a more standardised metabolic performance which then enables for example a more reliable in vitro predictive toxicological evaluation of candidate drugs prognosis of the cell culture before going into expensive clinical trials or similar, i.e. it results in a more reliable “filter” prior to embarking on animal or clinical trials.

At least for certain incubators, several cell culture chamber devices or bioreactors, e.g. with different types and/or sizes/state of cells, are used in the incubator where they all typically are located in the same—closable—open space or cavity. Even if provided with internal lighting, use in an incubator will reduce individual visibility of the content of each cell culture chamber device or bioreactor, often prompting users to repeatedly open and close—over time—the incubator and e.g. take out a cell culture chamber device or bioreactor for closer manual inspection. Repeatedly, opening and closing the incubator may at least increase the risk of contamination and at least temporarily disrupt the controlled internal environment of the incubator. More specifically, opening and closing the incubator disturb the internal environment (e.g. temperature, humidity, CO₂, etc.). Even when the door is not opened and closed there will be a tendency that warm air rises, i.e. the atmosphere at the top will be warmer than at the bottom. Therefore if high atmospheric uniformity is preferred or needed for high performance and reproducibility, it is advantageous to mix the internal atmosphere by some sort of recirculation. This however, increases the risk for contamination e.g. due to spores or bacteria laying at the bottom of the incubator chamber will be whirled around and come into contact with a contained cell culture chamber device and/or its culture chamber/enclosure. Accordingly, it would be a benefit to provide an incubator facilitating reduced need for opening and closing the incubator, and furthermore it would be a benefit to reduce the risk of contamination.

Furthermore, it is generally desirable to be able to decontaminate an incubator, and more specifically its space or cavity for containing the cell culture chamber devices or bioreactors, as well and as efficiently as possible to at least reduce the risk of contamination.

Accordingly, it would be an advantage to provide an incubator addressing one or more of the above mentioned drawbacks, at least to an extent. In particular, it would be an advantage to provide an incubator reducing the risk of contamination of the incubator space or cavity. It would also be an advantage to provide an incubator reducing a need for opening and closing it. It would be a further or additional advantage to provide an incubator enabling enhanced viewing of any contained cell culture chamber devices, and in particular of the content (in respective enclosures) of any contained cell culture chamber devices.

### SUMMARY

It is an object to provide an incubator addressing one or more of the above mentioned drawbacks, at least to an extent. It is a further object to provide an incubator reducing the risk of contamination of the incubator space or cavity and/or providing enhanced viewing of the content of any contained cell culture chamber devices or their respective enclosures (i.e. the cell culture media contained therein).

According to a first aspect, one or more of these objectives is achieved, at least to an extent, by an incubator configured to receive a predetermined number, at least one or a plurality, of cell culture chamber devices (also equally referred to herein as bioreactors). The predetermined number of cell culture chamber devices may e.g. be 1, 2, 3, 4, 5, 6 or more. Each (or at least one or some of the) cell culture chamber device(s) comprises an enclosure (also equally referred to herein as a cell chamber) configured to contain a cell culture media (typically, at least in use, containing cells). The incubator furthermore comprises
a housing comprising an incubation chamber configured to contain at least a respective part of the cell culture chamber devices when received by the incubator, and
at least one registration and/or detection device being integrated with the incubator and being configured to register and/or detect an illumination or visualisation signal after passing, reflecting, or propagating through at least a part of the enclosure of at least one of the predetermined number of cell culture chamber devices when received by the incubator.

In this way, monitoring (and/or other registration and/or detection) is readily enabled where the monitoring e.g. may be local or even remote as further disclosed herein. The illumination or visualisation signal may e.g. be a visual light signal and the registration and/or detection device(s) may e.g. be cameras or the like configured to providing a video feed or video capture, (e.g. periodic) still images, etc. of the content of the enclosure(s) of any contained cell culture chamber devices. Alternatively, the illumination or visualisation signal is a different signal, e.g. as disclosed herein.

The viewing and monitoring of the content of the enclosure(s) in this way significantly reduces the need for repeatedly opening and closing the incubator that in turn significantly reduces the risk of contamination of the incubation chamber. Furthermore, opening or accessing the incubation chamber also disrupts the controlled environment therein (e.g. with respect to temperature, humidity, and/or CO₂, etc.). In certain known types of incubators it has for example been shown that after opening the door to the incubation chamber for only about 30 seconds it took about 6 minutes to re-establish the proper temperature and CO₂ level again.

Additionally, the registration and/or detection device(s) may readily provide enhanced viewing (e.g. enlarged/zoomed, IR, etc. views) compared to manually looking through a glass panel or the like into the incubation chamber (even if lighting is present in the incubation chamber). Often a user would have to access the incubation chamber for an inspection or even having to stop the operation of the incubator and take out a cell culture chamber device for closer inspection, which could be detrimental to the content of the cell culture chamber device (e.g. as it is not rotated for a period of time and can be shaken or disturbed somewhat even if handled carefully).

Traditionally, when opening or accessing the incubation chamber, e.g. for inspection, the cell culture chamber devices and their enclosures are exposed to light, which potentially is detrimental for certain types of cells and spheroids. As mentioned in the following, the incubator could comprise one or more light sources for illuminating the incubation chamber. This could selectively be switched on when inspecting or documenting the content of the incubation chamber (the content of the cell culture chamber device(s)) using the registration and/or detection device(s), e.g. cameras, and switched off after, which would reduce the period of time of light exposure. Additionally, a user may inspect, document, etc. the content of multiple cell culture chamber devices faster thereby reducing the extent of light exposure.

In some embodiments, the incubation chamber is configured to contain the cell culture chamber devices in full (when received by the incubator). Alternatively, the incubation chamber is configured to contain only a part of the cell culture chamber devices, in particular at least a part, e.g. the whole, of the respective enclosure(s). In some embodiments, the cell culture chamber devices may e.g. be perfusion bioreactors (than can be self-sustained for prolonged periods of time e.g. up to about 14 days or more), comprising respective fresh and spent media reservoirs, drive element(s), etc. and it would be an advantage to have certain parts or components, such as the fresh and spent media reservoirs, the drive element(s), etc., outside the incubation chamber thereby reducing the risk of contamination and enabling easier cleaning. See e.g. Applicant's co-pending PCT patent application with application number PCT/EP2020/068632 for examples of perfusion cell culture chamber devices or bioreactors.

In some embodiments, the incubator further comprises at least one rotational drive unit, each rotational drive unit (or at least some of them)
configured to, e.g. or preferably releasably, receive at least one of the cell culture chamber devices, and
configured to rotate a cell culture chamber device, received by the rotational drive unit, about (at least) a predetermined rotational axis, e.g. or preferably a horizontal axis, of the received cell culture chamber device.

The rotational drive unit(s) is respectively configured to rotate one (or more) received cell culture chamber device(s) about one, two, or three mutually substantially perpendicular axes. Incubators rotating about two or three such axes are sometimes also referred to as so-called random positioning machines. In some embodiments, the predetermined rotational axis is a predetermined central axis of the received cell culture chamber device. Alternatively, the predetermined rotational axis is a predetermined central axis of the enclosure of the received cell culture chamber device. In some embodiments, the central axis of the received cell culture chamber device may be the same or coincide with the central axis of the enclosure of the received cell culture chamber device, i.e. if the enclosure is centrally arranged within the cell culture chamber device (which does not always need to be the case).

In some embodiments, the rotational drive unit(s) is/are clinostat drive unit(s).

In at least some (preferred) embodiments, the at least one registration and/or detection device is/are arranged aligned horizontally/lengthwise with the at least one cell culture chamber devices, e.g. aligned in a direction of the predetermined rotational axis or an axis substantially parallel thereto.

The at least one registration and/or detection device can in principle (in some embodiments) be located outside the incubator instead of being integrated with it, where the incubator then comprises a number of transparent windows or the like aligned with the cell culture chamber device(s) (when received by the incubator) allowing the registration and/or detection devices to register and/or detect the content of any received cell culture chamber device(s) when properly arranged.

In some embodiments, the incubator comprises an openable and closable door or lid (or other access element) and the incubation chamber comprises at least one incubation chamber wall, the at least one incubation chamber wall and the door or lid (or other element), when closed, defining the incubation chamber at least in part. In some embodiments, the at least one incubation chamber wall and the door or lid (or other access element), when closed, defines the incubation chamber in full. In some embodiments, the incubation chamber comprises only a single incubation chamber wall. In some further embodiments, the (single) incubation chamber wall is preferably generally ‘bowl’- or ‘pan’-shaped or generally hemispherical or semi-elliptical e.g. with a cylindrical section (having a circular cross-section substantially perpendicular to a first or length wise, e.g. horizontal, direction) so the incubation chamber does not have any sharp corners or edges thereby facilitating easy and efficient cleaning of the incubation chamber, which is significant for keeping the incubation chamber in an at least decontaminated (if not completely sterile) state. It is noted, that an incubator comprising a ‘rounded’ incubation chamber as described above (having a circular cross-section substantially perpendicular to the first or length wise, e.g. horizontal, direction) may be provided without any registration and/or detection device(s) and may be used independently thereof.

In some embodiments the incubator further comprises one or more locking, latching, or securing elements for the door or lid, e.g. of the type providing a so-called push-to-open and push-to-close locking functionality enabling hands-free opening and closing by a user in turn reducing the risk of contamination of the incubation chamber. Alternatively, the elements are of another type, e.g. touch-free, remotely operated, etc.

In some further embodiments, a first or an inner side (i.e. the side facing the incubation chamber when the door, lid, etc. is closed) of the door or lid (or other access element) comprises the at least one registration and/or detection device where the at least one registration and/or detection device is/are arranged facing so that an enclosure of at least one received cell culture chamber device is within a field of view of registration and/or detection of at least one of the at least one registration and/or detection device, i.e. the enclosure(s) of any received cell culture chamber device(s) is within the field of view of at least one registration and/or detection device.

In some embodiments (with a plurality of registration and/or detection devices), the registration and/or detection devices are arranged equidistantly in a substantially circular pattern in the door, lid, or the like of the incubator.

In some embodiments, the incubator comprises the predetermined number of registration and/or detection devices, i.e. one registration and/or detection device for each cell culture chamber device that can be received by the incubator or in other words in a one to one relationship. In some further embodiments, each registration and/or detection device is arranged so that a central axis of a field of view of registration and/or detection of a respective registration and/or detection device at least substantially aligns with a central axis of a respective enclosure of a received cell culture chamber device. In this way, one registration and/or detection device is dedicated to register and/or detect an illumination or visualisation signal for one specific cell culture chamber device, which typically will enhance the quality of the respective illumination or visualisation signals and/or also enhance the registration and/or detection (e.g. viewing) of the specific culture chamber devices, and in particular of the content of any contained cell culture chamber devices. Additionally, it will also be assured (or at least greatly facilitated) that the registration and/or detection device(s) and the cell culture chamber device(s) are correctly aligned (in X, Y, and Z dimensions), which is particularly significant given the typical short distances between them in such setups. Alternatively, one registration and/or detection device is dedicated to register and/or detect an illumination or visualisation signal for a plurality of specific cell culture chamber devices thereby reducing the number of registration and/or detection devices needed.

In some embodiments, the at least one registration and/or detection device is/are an imaging or vision system or device and the illumination or visualisation signal is ultraviolet, visible, infrared, and/or near-infrared light (and accordingly the at least one registration and/or detection device is/are configured to register or detect ultraviolet, visible, infrared and/or near-infrared light).

In some further embodiments, the incubator further comprises one or more light (or illumination) sources configured to illuminate at least a respective part of the cell culture chamber devices, e.g. illuminating at least a part of respective enclosure of any received cell culture chamber devices. In yet further embodiments, the one or more light (or illumination) sources is/are configured to respectively illuminate at least a first end, or a part or window thereof, of an enclosure of one or more cell culture chamber devices received by the incubator. In some further embodiments, the one or more light sources is/are arranged in the door or lid facing the one or more cell culture chamber devices when received by the incubator. Accordingly, (primarily) ‘front’-illumination of the enclosure(s) is/are provided, where ‘front’-illumination is to be taken as illumination originating from the opening of the incubation chamber toward the cell culture chamber device(s) (when received).

In some embodiments, at least one respective drive unit (e.g. one, some or all) of the at least one rotational drive unit comprises one or more light or illumination sources configured to illuminate at least a respective part of the cell culture chamber devices, e.g. illuminating at least a part of respective enclosure of any received cell culture chamber devices. In yet further embodiments, the one or more light or illumination sources is/are configured to respectively illuminate at least a second end, or a part or window thereof, of an enclosure of one or more cell culture chamber devices received by the respective drive unit.

In some alternative embodiments, the at least one respective drive unit (e.g. one, some or all) of the at least one rotational drive unit comprises a hollow rotational shaft comprising a light guide or other light or illumination element configured to illuminate at least a respective part of a cell culture chamber device or an enclosure of such when received by the drive unit.

Accordingly, (primarily) ‘back’-illumination of the enclosure(s) is/are provided, where ‘back’-illumination is to be taken as illumination originating opposite the opening of the incubation chamber and towards the cell culture chamber device(s) (when received).

In some embodiments, the at least one drive unit (or at least the ones providing back-lighting) comprises a cavity arranged adjacent to a received cell culture chamber device where the cavity comprises the one or more light or illumination sources. This provides a very compact drive unit with back-lighting.

In some embodiments, the one or more light or illumination sources are arranged offset from a central axis or rotational axis of the enclosure or of cell culture chamber device, which may provide simple/simpler access to the enclosure and thereby it's content.

Embodiments of the incubator may comprise a mix of front-illuminating and back-illuminating light or illumination sources, increasing the quality of any obtained detection and/or registration signals. Embodiments may alternatively comprise only one or more back-illuminating light or illumination light sources or comprise only one or more front-illuminating light or illumination light sources.

The light source(s) may e.g. be LED light source(s), laser light source(s), LED emitting laser light sources, or any other suitable light source.

Alternatively, another (one or more) illumination or visualisation signal source(s) is/are used instead of light sources being configured to emit another type of illumination or visualisation signal, e.g. through a second end, or part or window thereof, into a respective enclosure, wherein the at least one registration and/or detection device is configured to capture at least a part of the other type of illumination or visualisation signal transmitted through a first end, or part or window thereof, to outside the enclosure. This other type of registration and/or detection device(s) may e.g. be configured for registration of sound or acoustic waves (e.g. ultrasound) or for registration of electromagnetic radiation different than light (e.g. x-rays).

In the illustrated embodiments, the four light sources 701 are offset from a centre axis or rotational axis (see e.g. 260 in FIG. 5) of the enclosure or cell culture chamber device, which may provide simple access to content of the enclosure.

The light source(s) 701 may e.g. be LED light source(s) or any other suitable light source.

In some embodiments, the drive unit comprises a plurality of light sources, they may be of the same type or alternatively of different types (emitting different wavelengths).

In some further embodiments, the drive unit comprises a plurality of light sources being of at least two different types, where the at least two different types may be selected from the group of UV, visible, near IR, and IR light.

In some embodiments, the drive unit comprises a light diffusor arranged in a light propagation path from the one or more light sources to or towards an enclosure of a received cell culture chamber device. In some further embodiments, the light diffusor is arranged in the propagation path adjacent to or at least near a received cell culture chamber device (e.g. adjacent to or near a second end of an enclosure). The light diffusor may e.g. be arranged in a cavity of the drive unit. The light diffusor will provide a more uniform lighting towards the enclosure and may therefore increase the quality of the backlighting and thereby the detection and/or registration signal of the registration and/or detection device(s).

In some embodiments, a respective motor part of the at least one rotational drive unit is/are located outside the incubation chamber and inside the housing of the incubator. This readily removes a typically heat generating component of the drive unit(s) from the incubation chamber thereby making the temperature of the environment of the incubation chamber much easier to control and maintain and furthermore reduces the risk of inadvertently increasing/‘spiking’ the temperature in the incubation chamber, which may be detrimental to content of the cell culture chamber device(s) located in the incubation chamber. Another significant advantage is that it accordingly becomes easier to keep the incubation chamber clean, which is significant in order to avoid unwanted contamination of the received cell culture chamber device(s) by viruses, microorganisms of various types, etc. Furthermore, the incubator will typically consume less power since there is no need to actively remove generated heat from the incubation chamber. The generated heat in the housing may e.g. be removed passively.

It is noted, that an incubator with drive units with a respective motor part located outside the incubation chamber and inside housing of the incubator may be provided without any registration and/or detection device(s) and may be used independently thereof.

In some embodiments, the incubator further comprises a fan or ventilation unit (e.g. or preferably a rotating fan or ventilation unit) arranged in the incubation chamber and configured to cause an air flow inside the incubation chamber in response to a control signal. The presence of a fan or ventilation unit in the incubation chamber (or a fan or ventilation unit arranged elsewhere in the housing but still causing an air flow in the incubation chamber) promotes a uniform environment within the incubation chamber that typically is advantageous in relation to providing uniform humidity, uniform temperature, etc. that may be critical parameters to control in connection with the use of the incubator. Additionally, the fan or ventilation unit also helps in rapidly re-establishing a uniform environment after the door, lid, etc. have been opened and closed.

In some embodiments, the fan or ventilation unit is located generally centrally in the incubation chamber (at or towards a bottom or end wall of the incubation chamber), which further promotes the efficiency of the fan or ventilation unit, in particular in combination with an incubation chamber shaped as described above and elsewhere (bowl, hemispherical, etc.). For some embodiments, e.g. where the cell culture chamber devices are arranged in a substantially circular pattern in the incubation chamber, a central space is readily available for such a central fan or ventilation unit enabling a compact design.

In some further embodiments, the central fan or ventilation unit is located ‘behind’ the cell culture chamber devices (when received), i.e. between the cell culture chamber devices and a bottom or end wall of the incubation chamber.

The incubator may comprise two or more (e.g. smaller) fan or ventilation units instead of a single fan or ventilation unit e.g. arranged in another manner.

It is noted, that an incubator with an fan or ventilation unit arranged in the incubation chamber and being configured to cause an air flow inside the incubation chamber in response to a control signal (and embodiments thereof) may be provided without any registration and/or detection device(s) and may be used independently thereof.

In some embodiments, the incubator further comprises an arrangement for exposing the incubation chamber directly with UVC light to decontaminate it such as one or more UVC light sources. It is noted, that this is different (and typically more efficient with relation to decontamination) that having an arrangement that ventilates or otherwise transfer air from the incubation chamber to and past an area being exposed to UVC light. Such a traditional UVC light area would typically be located elsewhere in the housing of the incubator and would typically require forced ventilation via a duct or similar and expose much less air.

Having the UVC light arrangement located inside the incubation chamber, thereby exposing the interior of the incubation chamber directly with UVC light, works especially well with an incubation chamber having a general cylindrical cross section (substantially perpendicular to a first or length wise direction, which often will be a horizontal direction) with no sharp corners or edges (as e.g. a generally square incubation chamber typically will have), e.g. such as an incubation chamber shaped as described above and elsewhere (bowl, hemispherical, etc.).

UVC light should be emitted in the incubation chamber when there is no cell culture chamber devices present or then only with the cell culture chamber devices that have a UVC light shielded enclosure for containment of cell culture.

In some embodiments, the UVC light(s) is/are rotating UVC light(s) configured to rotate about a predetermined axis (e.g. about the first or length wise direction or an axis parallel thereto, which often will be a horizontal direction) within the incubation chamber thereby sweeping the interior of the incubation chamber increasing the effect of the UVC light (in particular in connection with an incubation chamber shaped as described above and elsewhere (bowl, hemispherical, etc.)).

In some further embodiments, the UVC light(s) is/are asymmetrical, i.e. emits UVC light in (at least) two different, e.g. opposed, directions working particularly well in connection with rotation.

A UVC light source may e.g. be located generally centrally in the incubation chamber (at or towards a bottom or end wall of the incubation chamber).

In some embodiments, a rotating UVC light source or similar is integrated with a rotating fan or ventilation unit thereby providing a very compact and efficient design. Furthermore, the UVC light is thereby rotated together with the fan or ventilation unit thereby efficiently sweeping the incubation chamber. Preferably, the activation of the UVC light may be done independently of activation of the fan or ventilation unit, i.e. it should be possible to rotate the fan or ventilation unit (and thereby rotate the integrated UVC light) but without emitting UVC light.

In some other embodiments, the incubator comprises an (e.g. rotating) UVC arrangement/a UVC light and no fan or ventilation unit (rotating or otherwise). In some further embodiments, the incubator comprises a fan or ventilation unit (rotating or otherwise) and no UVC arrangement/a UVC light.

In some embodiments, at least a part of an inner surface of the incubation chamber (e.g. at least a part of the at least one incubation chamber wall and/or the first or inner side or surface of the door, lid, etc.) comprises a UVC reflecting material or coating, which typically will increase the effect of the UVC light.

Additionally, such sweeping UVC illumination inside the incubation chamber will also be fairly uniformly distributed in the incubation chamber and if the incubation chamber comprises a UVC reflecting material or coating, then UVC light will better reach regions of the incubation chamber not directly irradiated. Both of these factors enables keeping the UVC irradiation dose as low as possible while still achieving a required level of decontamination.

It is noted, that an incubator with an UVC arrangement located inside the incubation chamber and being configured to expose the interior of the incubation chamber directly with UVC light (and embodiments thereof) may be provided without any registration and/or detection device(s) and may be used independently thereof.

In some embodiments, the incubator further comprises at least one heating element configured to controllably heating the inside of the incubation chamber in response to a control signal. In some further embodiments, the heating element comprises a back or main part substantially matching an e.g. planar shape of the incubation chamber wall that forms a ‘back’ or ‘end’ wall of the incubation chamber and furthermore comprises a number of heating ‘arms’, ‘flaps, or ‘tongues’ extending to the side/sides of the incubation chamber wall thereby effectively increasing the area of the incubation chamber wall that may be directly exposed to heating in turn increasing the possible heating speed and homogeneity of the inside of the incubation chamber. The heating element may be any suitable heating element, preferably a relatively flat or thin element, such as a heating foil, grid, etc. In some embodiments, the heating element is a self-adhesive heating element. Alternatively, it may be secured to the incubation chamber wall in another suitable way. In at least some embodiments, the heating element is located on an exterior of the incubation chamber wall, i.e. on the surface opposite the surface inside the incubation chamber.

A heating element together with a fan or ventilation unit, e.g. as disclosed herein, causing an airflow inside the incubation chamber enables a very uniform heating distribution within the incubation chamber, which is significant in order to effectively and quickly being able to control the temperature.

In some embodiments, the incubator further comprises
a one or more processing units,
an electronic memory and/or electronic storage, and
one or more signal transmitter and receiver communications elements configured to communicate with a network,
   wherein the one or more processing units is/are configured to communicate via the network with at least one external computational device, e.g. one or more of a user interface device, a client and/or server computer or device, a network connected storage device, and/or one or more additional incubators.

In some embodiments, captured or obtained videos and/or pictures and/or other detection and/or registration signals of the registration and/or detection device(s) may be transmitted by the incubator to the external computational device, e.g. for presentation (e.g. remote viewing or remote online viewing), storage, and/or further digital processing.

A user interface device may e.g. be configured for online monitoring of the signals obtained by the registration and/or detection device(s) of the incubator.

In some embodiments, the incubator is further configured to receive, via the network, user input control data obtained by a user interface device and/or another external computational device (e.g. client, server, master, etc.), and to change or adapt operation in response to at least a part of the received user input control data.

In some further embodiments, the incubator (e.g. a master unit) is configured to receive further user input control data and communicate, at least a part thereof, to another incubator (e.g. a slave unit), wherein the other incubator is configured to change or adapt operation in response to at least a part received further user input control data.

Additional embodiments of such an incubator is disclosed herein, e.g. in connection with FIG. 16.

In some embodiments, the incubator comprises a (at least one) cell culture chamber device for the growing of cell cultures and tissues, where the cell culture chamber device comprises an enclosure configured to contain a typically aqueous cell culture media. The cell culture chamber device further comprises a first end, a second end, and at least one connecting (e.g. circumferential) wall connecting the first and the second ends. The first end, the second end, and the at least one connecting wall at least in part defines the enclosure. The enclosure may e.g. also be referred to as a cell culture enclosure, cell chamber, or etc. The first end may e.g. also be referred to as a first end segment or first part of the enclosure and the second end may e.g. also be referred to as a second end segment or second part of the enclosure. The first end may e.g. also be referred to as a viewing end or part, or as a primary viewing end or part. The first end, or a part or window thereof, is substantially transparent. The second end and/or at least one of the at least one connecting wall, or a respective part or window thereof, is/are substantially transparent or is/are substantially translucent. The first end or the part or window thereof is configured to be optically or otherwise (e.g. or i.e. with respect to other electromagnetic radiation or mechanical waves such as sound or acoustic waves) aligned (at least for some period of time or periodically) with the second end or the part or window thereof and/or with at least one of the at least one connecting wall or the part or window thereof so that light or another illumination or visualisation signal, transmitted through or by the second end or the part or window thereof and/or through or by the at least one of the at least one connecting wall or the part of window thereof into the enclosure, is transmitted or propagates through at least a part of the cell culture media and out through the first end or the part or window thereof to outside the enclosure, and e.g. to outside the cell culture chamber device. It is noted (for relevant embodiments), that the first end (or the part or window thereof) does not need to be optically or otherwise aligned with the second end (or the part or window thereof) by being across or directly across each other, even though that provides a very expedient way of providing this. For example, a suitable optically based or other electromagnetically radiation based, sound/acoustic wave based, etc. system or one or more suitable devices or components (e.g. reflectors, mirrors, sound or light-guides, etc.) could be used to align the respective ends (or parts/windows) at least during some time. What is significant in a broadest sense is that light or another illumination or visualisation signal passes through a part or a significant part of the cell culture media in the enclosure and afterwards is emitted outside the enclosure in an as unobstructed way as possible or necessary (apart from being influenced by the content of the cell culture media) allowing registration and/or characterisation of part or all of the contents of the enclosure. In some embodiments, the first end (or at least the part or window thereof) is substantially planar as this provides an undistorted optical image or projection. However, the ends (or parts or windows) may be curved, at least to some extent. The second end and/or the connecting wall (or respective part(s) or window(s) thereof) may be curved but in some embodiments, the second end and/or the connecting wall is (or respective part(s) or window(s) thereof) is/are substantially planar.

In this way, a cell culture chamber device is provided having un-obstructed (apart from being influenced by the content of the cell culture media) light or other illumination or visualisation signal propagation paths propagating through at least a part of any cell culture media contained in the enclosure. It also enables the provision of back-light or emission of another illumination or visualisation signal from ‘behind’, i.e. light shone or another illumination or visualisation signal emitted through the second end and/or the connecting wall(s) (e.g. towards the second end), greatly enhancing visual inspection (manual or automatic) from the other/opposite side (i.e. via the first end). This is particularly useful e.g. for inspection of several cell culture chamber devices arranged in an incubator or the like. If the second end is transparent, then visual or other inspection (e.g. acoustic or electromagnetic radiation different from light), manual (in case of light) and/or automatic (in case of light or other electromagnetic radiation or sound or acoustic waves using a suitable sensor), is furthermore enabled from both ends, i.e. two sides, of the enclosure. In some embodiments, all the parts of the cell culture chamber device are transparent.

Such a cell culture chamber device is furthermore especially useful for use in connection with an incubator according to the first aspect and as disclosed herein, since the transparent first end (and transparent) allows for increased quality of detection and/or registration of its content by one or more one registration and/or detection devices as disclosed herein. As mentioned, the incubator according to the first aspect and as disclosed herein is configured to receive one or more of such cell culture chamber devices.

The light is at least in some embodiments natural or artificial light or a combination thereof, typically or preferably visible light having a wavelength of about 400 to about 700 nanometres or at least a sub-range thereof. Alternatively, the light could e.g. be infrared or near-infrared light respectively having a wavelength of about 700 nanometres to about 1 millimetre or about 900 nanometres to about 2500 nanometres. As yet another alternative, the illumination or visualisation signal is an electromagnetic radiation having a wavelength different from visible light or light, e.g. an infra-red or x-ray signal. As a further alternative, the illumination or visualisation signal is a sound or an acoustic wave signal, e.g. ultrasound. By (substantially) transparent and (substantially) translucent is meant that the ends or walls (or respective parts or windows thereof) are sufficiently (substantially) transparent and/or sufficiently (substantially) translucent in relation to the type of light or other illumination or visualisation signal intended to be used with the cell culture enclosure/the cell culture chamber device.

In some embodiments, the cell culture chamber device (and the enclosure) is configured (or at least suitable) for rotation about a (at least one) predetermined rotational axis, e.g. as generally known. In some further embodiments, the cell culture chamber device (and the enclosure) is configured for clinostat rotation or for rotation negating or supplementing, at least to a certain extent, the effects of gravitational pull on content in the cell culture chamber device or more specifically the content in the enclosure. The cell culture chamber device may e.g. comprise one or more attachment or connection elements for, preferably but not necessarily, releasably attaching or connecting with a drive unit.

In some embodiments, the first end or the part or window thereof and the second end or the part or window thereof are opposite each other in a predetermined direction, e.g. along a central and/or length-wise axis of the enclosure and/or the cell culture chamber device where the axis extends between the first end or the part or window thereof and the second end or the part or window thereof. In some further embodiments (where the cell culture chamber device is configured for rotation as mentioned elsewhere), the central axis may also be the axis about which the cell culture chamber device is rotated or at least is rotatable.

In at least some embodiments, the enclosure is symmetrically located in the cell culture chamber device with respect to the axis of rotation/the central axis.

In some embodiments, a material or a group of materials of one or more predetermined parts, e.g. all parts, of the enclosure and/or of the cell culture chamber device is or are opaque to UVC light (i.e. light having a wavelength range of about 100 to about 280 nanometres) where the one or more predetermined parts are configured so no or substantially no UVC light can reach inside the enclosure. In this way, it is possible to expose the whole cell culture chamber device to UVC light (using the well-known disinfecting and sterilising properties of the UVC light) without detrimental effect to the content inside the enclosure of the cell culture chamber device. In some further embodiments, the UVC opaque material or group of materials is/are or comprises an UVC opaque plastic as generally known. See e.g. https://www.gsoptics.com/transmission-curves/ and for examples of UVC absorbing plastics, in particular UVC opaque plastic such as polycarbonate, polystyrene, poly(methyl methacrylate) (PMMA—commonly known as acrylic or plexiglass), polyester (e.g. OKP4) or polyetherimide (e.g. Ultem) or UVC absorbing additives https://polymer-additives.specialchem.com/product-categories/additives-light-stabilizers-uv-absorbers (see https://polymer-additives.specialchem.com/product-categories/additives-light-stabilizers-uv-absorbers), including but not limited to Tinuvin®, Uvasorb®, ADK STAB or Cel-Span®.

In some embodiments, the cell culture chamber device further comprises a circumferential gas exchanger
arranged circumferentially about or along at least a part of the enclosure or about a central or lengthwise axis of the cell culture chamber device (typically the central or lengthwise axis of the cell culture chamber device and the central or lengthwise axis of the enclosure, as disclosed herein, will coincide or at least be substantially parallel), and e.g. or preferably about the predetermined rotational axis (if the cell culture chamber devices is configured for rotation), and
comprising a cavity comprising (or defining) a volume connecting a gas exchange interface of the enclosure with ambient air or gas of the cell culture chamber device.

That the gas exchanger is circumferential (and other relevant elements designated herein to be circumferential, e.g. a circumferential humidifier) is to mean that the gas exchanger is arranged as radially surrounding at least a part of the enclosure. For an enclosure with a circular cross section substantially perpendicular to the central and/or lengthwise axis and a circumferential/radially surrounding gas exchanger, the cross section of both (substantially perpendicular to the central and/or lengthwise axis) would produce an inner circle (being a surrounded part of the enclosure) and an outer surrounding ring (being the gas exchanger). See e.g. FIG. 10 for an example of this according to the illustrated embodiment. In this way, the gas exchanger is arranged off centre lengthwise (but typically still about the central and/or rotational axis) and away from the central and/or lengthwise axis (typically extending between the first and the second ends and being substantially parallel to the rotational axis), i.e. the gas exchanger is not ‘stacked’ next to the enclosure or any other component in a lengthwise direction but rather radially surrounding or being located around an exterior of the enclosure. This enables for much more efficient back-lighting or other illumination or visualisation by another type of illumination or visualisation signal as the gas exchanger no longer obstructs light or the illumination or visualisation signal from one or more light or illumination or visualisation signal sources located at or near the second end. Additionally, the gas exchanger will at least obstruct light or illumination or visualisation signal from one or more light or illumination or visualisation signal sources located at or near the at least one connecting wall (e.g. towards the second end) to a lesser degree.

Additionally, by having a circumferential gas exchanger, the lengthwise extent of the cell culture chamber device is also greatly reduced reducing the lengthwise ‘footprint’/form-factor which may be beneficial for design considerations and shortens the light-path or path of the other illumination or visualisation signal.

It is noted, that the provision of such a circumferential gas exchanger functions particularly well with an incubator according to the first aspect (as it is offset/off-centre and thereby obstructs light or another illumination or visualisation signal at least to a lesser degree).

In some embodiments, the gas exchange interface is or comprises a circumferential gas permeable membrane, e.g. a semipermeable membrane, either porous or non-porous, configured to exchange gases, such as oxygen and carbon dioxide, with an inside and/or content of the enclosure, where the circumferential gas permeable membrane is arranged circumferentially along a circumferential part of the enclosure.

In some embodiments, the circumferential gas permeable membrane constitutes at least a part, e.g. or preferably all, of at least one of the at least one connecting wall of the enclosure. Accordingly, the first and the second ends together with gas permeable membrane defines the enclosure, at least in part. It is noted, that the circumferential gas permeable membrane does not need to take up a full circumference.

In some embodiments, the gas exchange interface or the circumferential gas permeable membrane is supported by at least one support structure, e.g. a grid like support structure, comprising a number of openings configured to connect the gas exchange interface or the circumferential gas permeable membrane with air or gas of the volume of the cavity of the circumferential gas exchanger.

In some embodiments, the circumferential gas exchanger is connected with the ambient air or gas of the cell culture chamber device via at least one gas or air inlet and/or outlet.

In some embodiments, at least one of the at least one gas or air inlet and/or outlet is a double vent or port configured to, e.g. or preferably simultaneously, draw in ambient air or gas into the cavity of the circumferential gas exchanger and expel air or gas out of the cavity of the circumferential gas exchanger in response to the cell culture chamber device being rotated thereby creating an air flow. The double vent or port may e.g. be configured to operate according to the Coand\check{a} effect or principle. In at least some such embodiments, mirrored but otherwise symmetric vents or ports constituting the double vent or port enables draw in and expel air or gas both in clockwise and counter clockwise rotation of the cell culture chamber device (just with reversed resulting air flow) resulting in equal rates of gas exchange when rotated in either direction (at the same speed). In some further embodiments, the degree of air movement or flow can be regulated by regulating the respective sizes of the vents of the double vent for example with a slider (e.g. regulating between 0 to about 100% of maximum air flow) or differently sized plugs (e.g. plugs for ⅓, ⅔, 3/3 of maximum air flow), or in another suitable manner.

In some embodiments, the cell culture chamber device further comprises a circumferential humidifier
arranged circumferentially about at least a part of the enclosure or about a central and/or lengthwise axis of the cell culture chamber device, e.g. or preferably about the predetermined rotational axis (if the cell culture chamber device is configured for rotation), and
comprises or is connected to one or more liquid or moisturising reservoirs or elements configured to humidify or moisturise air or gas in at least a part of the cavity of the circumferential gas exchanger or of the air flow.

It is noted, that the provision of such a circumferential humidifier functions particularly well with an incubator according to the first aspect (as it is offset/off-centre and thereby obstructs light or another illumination or visualisation signal at least to a lesser degree).

In some embodiments, the one or more liquid or moisturising reservoirs or elements is/are configured to humidify or moisturise air or gas in the vicinity of or being adjacent to at least a part of the gas exchange interface or the circumferential gas permeable membrane, the part being outside the enclosure.

In some embodiments, at least one of the one or more liquid or moisturising reservoirs or elements comprises a liquid being either a sterile aqueous solution (or at least an initially sterile aqueous solution) or an aqueous solution containing one or more additives configured to maintain sterility and/or other compounds extending shelf life and/or a predetermined function or utility, e.g. a coloured dye to aid visualisation of remaining water content of the content of the enclosure. It is noted, that the sterile aqueous solution during use typically and eventually will become non-sterile.

In some embodiments, at least one of the one or more liquid or moisturising reservoirs or elements comprises a water or solute-containing material such as a gel, sponge, or a particulate material (e.g. water- or aqua beads, slush powder or water gel powder (also referred to as “snow”), etc.). Water or aqua beads are sometimes also referred to as water crystal gel, hydrated water gel, or gel beads and is any gel that absorb and contain a relatively large amount of water. They are typically spherical and may e.g. be composed of a water-absorbing superabsorbent polymer (SAP, also known as slush powder in dry form) such as a polyacrylamide e.g. sodium polyacrylate.

In some embodiments, a material or a group of materials of the one or more liquid or moisturising reservoirs or elements and/or one or more predetermined parts of the cell culture chamber device is configured to allow transmission of UVC light to decontaminate a content of the one or more liquid or moisturising reservoirs or elements.

In some embodiments, the cell culture chamber device comprises a first or central housing (may in some embodiments also be referred to as face plate or similar) and a cover where the first or central housing comprises the second end and the cover comprises the first end, and wherein the first or central housing is configured to receive, e.g. releasably, the cover, where a cavity between the first or central housing and the cover is defined when the cover is received by the first or central housing, and where the (resulting) cavity (between the first or central housing and the cover) defines at least a part of the enclosure. In this way, the enclosure is provided (at least in part) in a particular expedient way.

In some embodiments, the cavity between the first or central housing and the cover comprises the circumferential gas permeable membrane constituting at least a part of the at least one connecting wall of the enclosure thereby connecting the first end of the cover with the second end of the first or central housing.

In some embodiments, the cell culture chamber device further comprises a main housing configured to receive the first or central housing and the cover. In some further embodiments, the main housing comprises an opening aligning with the second end of the first or central housing when the first or central housing is received by or in the main housing, where the size of the opening is substantially of the same size as the second end. Accordingly, the main housing (given the opening) will not block a line of sight to the second end. In some further embodiments, the main housing and the first or central housing comprises elements that fit tightly together obviating the need for sealing materials e.g. gluing, welding, compressible parts (e.g. o-rings), etc.

In some embodiments, the main housing and the first or central housing, when received by the main housing defines the cavity of the circumferential gas exchanger (if such is present), and the first or central housing comprises the double vent or port arranged to be substantially perpendicular to the predetermined rotational axis, e.g. on a front side or front facing side of the main housing.

In some embodiments, the first or central housing comprises at the least one (e.g. grid like) support structure, comprising a number of openings.

In some embodiments, the second end or the part or window thereof is substantially transparent (instead of substantially translucent) and the cell culture chamber device further comprises or is connected to a light diffusor (also referred to as optical diffusor) configured to receive light and to provide substantially uniform light to the second end or the part or window thereof thereby providing substantially uniform illumination of the cell culture media when contained in the enclosure. The light diffusor is located in the light propagation path between the light source (natural and/or artificial) and before the enclosure/the second end or the part or window thereof. The substantially uniform illumination of the cell culture media in this way readily enables (further) enhanced visual (manual or automatic) monitoring and thereby visual assessment of the content of the enclosure.

For alternative embodiments, where the second end or the part or window thereof is substantially translucent (instead of substantially transparent), the translucent end or part or window will effectively function as a light diffuser thereby saving the need for such an additional component. For further alternative embodiments, where the second end or the part or window thereof is substantially translucent (instead of substantially transparent), a light diffuser is still present, thereby in effect providing a double-diffusor (one by the translucent end or part or window thereof and one by the light diffuser) that may produce an even further uniform light distribution (at the ‘cost’ of some but typically not a lot of light energy).

In yet further alternative embodiments, the diffusor is not a light diffuser but a diffusor with respect to the other type of illumination or visualisation signal, e.g. an acoustic diffusor or a diffusor for electromagnetic radiation other than light.

In some alternative embodiments, the cell culture chamber device is configured for front-lighting (or other front-application of the other type of illumination or visualisation signal) either in addition to or as an alternative to back-lighting or emission of another illumination or visualisation signal from ‘behind’. In some such further alternative embodiments, the diffusor (if one is present) may be replaced by a suitable reflector, e.g. a parabolic reflector.

In some alternative embodiments, the cell culture chamber device is configured for side-lighting (or other side-application of the other type of illumination or visualisation signal) either in addition to or as an alternative to back- or front-lighting or emission of another illumination or visualisation signal from ‘behind’ or the ‘side(s)’.

In some embodiments, a respective cross section (each being substantially perpendicular to a central axis extending between the first and the second end) of the first end and/or the second end is/are substantially circular.

The overall shape of the cell culture chamber device is preferably such that it is sufficiently suitable for rotation about at least one axis. I.e. it should preferably avoid sharp (cross-sectional perpendicular to the axis of rotation) corners as this may introduce unwanted/irregular/too large shear forces, unwanted variations in the growth environment, or similar on growing cells or tissue during rotation, which could be detrimental to an optimal and/or uniform formation of for example spheroids.

In some further embodiments, the overall shape of the cell culture chamber device is (substantially) cylindrical with the first and second ends respectively forming the circular bases of the cylinder.

This provides a simple suitable shape readily enabling simple/simpler manufacturing of the cell culture chamber device. Furthermore such a generally cylindrical shape is also very suitable for being rotated about an axis, e.g. about its (lengthwise) central axis extending between the first end (or the part or window thereof) and the second end (or the part or window thereof).

In alternative embodiments, the overall shape of the cell culture chamber device is (substantially) spherical.

Alternatively, the cross sections of the first end and/or the second end are not circular but instead the cross sections (or one of them) may e.g. be an n'th level polygon where n is equal to or larger than three and preferably equal to or larger than at least six (i.e. an hexagon), e.g. equal to or larger than eight (i.e. an octagon) or more. Preferably, n is an even number as this promotes the symmetricity of the cell culture chamber device about a central or rotational axis (that may coincide) extending between the ends. A circular cross section is approximated to a larger and larger degree as n increases.

The cross sections of the first end and/or the second end may also be elliptical.

The cell culture chamber device may have a first extent (e.g. length) and at least a second extent (e.g. height, depth, or diameter) (see e.g. ‘L’ and ‘D’ in FIG. 8). In some embodiments, the first extent/length (L) is less than the second extent/height, depth, or diameter (D), i.e. the circumferential extent is larger than the lengthwise extent (for generally cylindrical shapes and similar). In some embodiments the ratio between the first extent/L and the second extent/D is about 1:1 to about 1:10. In some further embodiments, the ratio is about 1:2 to about 1:5 and in other further embodiments, the ratio is about 1:3 to about 1:4. These embodiments respectively provide a very (lengthwise) compact cell culture chamber. The circumferential design of the gas exchanger and/or the humidifier greatly enables a higher ratio and thereby a smaller (lengthwise) form-factor.

The cross sections (and/or the shapes) of the first end and the second end may be different from each other.

In principle, the cell culture chamber device might have any suitable regular or irregular shape (while supporting rotation as described herein) but it is preferred for manufacturing purposes if the shape is relatively simple.

In some embodiments, the enclosure and/or the cell culture chamber device further comprises one or more fiducial and/or identification markers, such as identification markings, barcodes, points of reference, etc. At least some of the fiducial and/or identification markers is/are preferably machine readable. This may e.g. be advantageously used in connection with monitoring using at least one registration and/or detection device as disclosed herein (see e.g. 220 in FIGS. 1, 2, and 5), e.g. an imaging or vision system or device. The fiducial marker(s) enables determination of the orientation of the cell culture chamber device (and e.g. in particular of the enclosure) for use with at least one registration and/or detection device as disclosed herein. An identification marker is preferably unique to the particular cell culture chamber device that it is comprised by.

In some embodiments, the cell culture chamber device further comprises one or more aligning elements (e.g. location bar and slit or slot, etc.) for aligning different parts of the cell culture chamber (e.g. for relevant embodiments aligning the cover with the first or central housing or the main housing).

In some further embodiments, an aligning element may also function in addition as a fiducial marker.

In some embodiments, the second end and/or at least one of the at least one connecting wall comprises one or more integrated light sources.

In some embodiments, the second end and/or at least one of the at least one connecting wall is/are or comprises a fluorescent light emitting element.

In some embodiments, the cell culture chamber device comprises a closable and/or sealable first port connected to the inside of the enclosure and a closable and/or sealable second port connected to the inside of the enclosure. In some further embodiments, the first port and the second port are arranged on or to separate sides of the cell culture chamber device.

According to a second aspect is provided an incubator system comprising
a first incubator, e.g. or preferably an incubator according to the first aspect and embodiments thereof,
at least a second incubator, e.g. or preferably an incubator according to the first aspect and embodiments thereof, and
a user interface device and/or a client and/or server computer or device and/or at least one other external computational device,
   wherein
the first incubator is configured as a master unit and the at least a second incubator is configured as a slave unit,
the master unit is configured to control communication and/or data exchange between the master unit and all slave units with the user interface device and/or the client computer or device,
the user interface device and/or the client computer or device is configured to obtain user input control data and communicate the user input control data to the master unit, and
the master unit is configured to change or adapt operation in response to at least a part of the received user input control data and/or communicating at least a part of the received user input control data to at least one slave unit, the at least one slave unit configured to change or adapt operation in response to at least a part received user input control data.

In some embodiments, one or more of the incubators comprises at least one registration and/or detection device as disclosed herein and being configured to register and/or detect an illumination or visualisation signal after passing, reflecting, or propagating through at least a part of the enclosure of at least one of the predetermined number of cell culture chamber devices when received by the respective incubator(s).

The illumination or visualisation signal may e.g. be a visual light signal and the registration and/or detection device(s) may e.g. be cameras or the like configured to providing a video feed or video capture, (e.g. periodic) still images, etc. of the content of the enclosure(s) of any contained cell culture chamber devices. Alternatively, the illumination or visualisation signal is a different signal, e.g. as disclosed herein.

The user interface device may e.g. be configured for online monitoring of the first and/or any of the at least a second incubator. In some embodiments, the user interface device is configured to display in a user interface e.g. on a screen, a video online feed or a latest single or series of pictures for one or more of each incubator as obtained by the incubator via its respective cameras if comprising such as disclosed herein. Additional data, such as current rotation speed, rotational direction, ID, etc. for each particular cell culture chamber device may also be obtained and transmitted to the user interface device e.g. to be displayed on the device together with the video or image(s) for a respective cell culture chamber device. In addition to data that is specific to the cell culture chamber device(s), data for the respective incubators may also be obtained and provided e.g. one or more of current temperature, current pH value, current humidity, current CO₂, O₂, and/or N₂ level(s), etc. of the respective incubation chambers of the humidifier(s) as obtainable by a number of appropriate sensors located appropriately in the humidifier(s). The humidifier(s) may e.g. also send an alert or alarm to the user interface device (or another connected external computational device) if certain one or more parameters is/are outside an acceptable range of values, above or below an accepted value, etc. (e.g. if the measured current temperature exceeds a given temperature threshold or value, etc.).

The online feed or the pictures may readily enable manual inspection of the state of contained spheroids, e.g. their size, their orbit, etc., which might prompt a user to want to change, e.g. increase, the rotational speed, if for example the spheroids now have become larger and thereby heavier (prompting for an increased rotational speed). The ID of a particular cell culture chamber device may e.g. be obtained automatically by capturing an image or video of one or more fiducial and/or identification markers or codes and performing appropriate image analysis or other digital processing. In a similar manner, the presence of bubbles and/or an actual volume of cell culture media contained in a specific enclosure of a cell culture chamber device may also be obtained and presented by capturing an image or video of a number of suitable level or fill-rate indicators.

In some embodiments, the incubator(s) and/or the user interface device is further configured to perform data logging and/or documentation e.g. collecting and storing data such as temperature, humidity level, rotational speed, e.g. over time and e.g. including averages as well as duration and number of pauses (without rotation), etc. for at least some, e.g. all, of the cell culture chamber devices. This may e.g. be supplemented with video(s) and/or still image(s). The data of the data logging or documentation may e.g. be stored (e.g. also) in a cloud computing environment.

In addition or alternatively, the above functionality is provided for other types of illumination or visualisation signal(s), e.g. as disclosed herein, than video and images.

Additional embodiments of such an incubator system is disclosed herein, e.g. in connection with the first aspect and/or FIG. 16.

According to a third aspect is provided an incubator configured to receive a predetermined number, at least one or a plurality, of cell culture chamber devices, each cell culture chamber device comprising an enclosure configured to contain a cell culture media, the incubator comprising
a housing comprising an incubation chamber configured to contain at least a respective part of the cell culture chamber devices (100) when received by the incubator, and
an UVC arrangement configured to expose an interior the incubation chamber directly with UVC light.

Having the UVC light arrangement located inside the incubation chamber, thereby exposing the interior of the incubation chamber directly with UVC light, works especially well with an incubation chamber having a general cylindrical cross section (substantially perpendicular to a first or length wise direction, which often will be a horizontal direction) with no sharp corners or edges (as e.g. a generally square incubation chamber typically will have), e.g. such as an incubation chamber shaped as described above and elsewhere (bowl, hemispherical, etc.).

In some embodiments, the UVC arrangement comprises one or more UVC lights arranged inside the incubation chamber.

In some embodiments, the UVC light(s) is/are rotating UVC light(s) configured to rotate about a predetermined axis (e.g. about the first or length wise direction or an axis parallel thereto, which often will be a horizontal direction) within the incubation chamber thereby sweeping the interior of the incubation chamber increasing the effect of the UVC light (in particular in connection with an incubation chamber shaped as described above and elsewhere (bowl, hemispherical, etc.)).

In some further embodiments, the rotating UVC light(s) is integrated with a rotating fan or ventilation unit.

In some embodiments, the UVC light(s) is/are asymmetrical, i.e. emits UVC light in (at least) two different, e.g. opposed, directions working particularly well in connection with rotation.

In some embodiments, the UVC light(s) is/are located generally centrally in the incubation chamber (e.g. at or towards a bottom or end wall of the incubation chamber).

In some embodiments, at least a part of an inner surface of the incubation chamber (e.g. at least a part of the at least one incubation chamber wall and/or the first or inner side or surface of the door, lid, etc. if present) comprises a UVC reflecting material or coating.

Aspects and embodiments of the UVC light(s) of the third aspect is, at least in some embodiments, the same or corresponding (with same or corresponding advantages for the same reasons) as the UVC light(s) and embodiments thereof as described herein in connection with the first aspect.

According to a fourth aspect is provided an incubator (200, 200′) configured to receive a predetermined number, at least one or a plurality, of cell culture chamber devices (100), each cell culture chamber device (100) comprising an enclosure (110) configured to contain a cell culture media, the incubator (200, 200′) comprising
a housing (210) comprising an incubation chamber (201) configured to contain at least a respective part of the cell culture chamber devices (100) when received by the incubator (200, 200′), where the incubation chamber (201) comprises a circular cross-section substantially perpendicular to a first or length wise, e.g. horizontal, direction.

In some embodiments, the incubation chamber (201) is preferably generally ‘bowl’- or ‘pan’-shaped or generally hemispherical or semi-elliptical e.g. with a cylindrical section (having the circular cross-section) so the incubation chamber does not have any sharp corners or edges thereby facilitating easy and efficient cleaning of the incubation chamber.

Aspects and embodiments of the incubation chamber of the fourth aspect is, at least in some embodiments, the same or corresponding (with same or corresponding advantages for the same reasons) as the incubation chamber and embodiments thereof as described herein in connection with the first aspect.

According to a fifth aspect is provided an incubator configured to receive a predetermined number, at least one or a plurality, of cell culture chamber devices, each cell culture chamber device comprising an enclosure configured to contain a cell culture media, the incubator comprising
a housing comprising an incubation chamber configured to contain at least a respective part of the cell culture chamber devices when received by the incubator, and
at least one rotational drive unit, each rotational drive unit
   configured to, e.g. or preferably releasably, receive at least one of the cell culture chamber devices, and
   configured to rotate a cell culture chamber device, received by the rotational drive unit, about a predetermined rotational axis of the received cell culture chamber device, the predetermined rotational axis e.g. being a predetermined central axis of the received cell culture chamber device or of the enclosure of the received cell culture chamber device,
      wherein a respective motor part of the at least one rotational drive unit is located outside the incubation chamber and inside the housing.

This readily removes a typically heat generating component of the drive unit(s) from the incubation chamber thereby making the temperature of the environment of the incubation chamber much easier to control and maintain and furthermore reduces the risk of inadvertently increasing/‘spiking’ the temperature in the incubation chamber, which may be detrimental to content of the cell culture chamber device(s) located in the incubation chamber. Another significant advantage is that it accordingly becomes easier to keep the incubation chamber clean, which is significant in order to avoid unwanted contamination of the received cell culture chamber device(s) by viruses, microorganisms of various types, etc. Furthermore, the incubator will typically consume less power since there is no need to actively remove generated heat from the incubation chamber. The generated heat in the housing may e.g. be removed passively.

Aspects and embodiments of the rotational drive unit(s) of the fifth aspect is, at least in some embodiments, the same or corresponding (with same or corresponding advantages for the same reasons) as the rotational drive unit(s) and embodiments thereof as described herein in connection with the first aspect.

According to a sixth aspect is provided an incubator configured to receive a predetermined number, at least one or a plurality, of cell culture chamber devices, each cell culture chamber device comprising an enclosure configured to contain a cell culture media, the incubator comprising
a housing comprising an incubation chamber configured to contain at least a respective part of the cell culture chamber devices when received by the incubator, and
a, e.g. rotating, fan or ventilation unit arranged in the incubation chamber and configured to cause an air flow inside the incubation chamber in response to a control signal.

Aspects and embodiments of the fan or ventilation unit of the sixth aspect is, at least in some embodiments, the same or corresponding (with same or corresponding advantages for the same reasons) as the fan or ventilation unit and embodiments thereof as described herein in connection with the first aspect.

The cell culture chamber devices of the third, fourth, fifth, and sixth aspect may correspond to cell culture chamber devices and embodiments thereof according to the first aspect and/or as disclosed herein.

Further details and embodiments are disclosed in the following.

### Definitions

All headings and sub-headings are used herein for convenience only and should not be constructed as limiting the invention in any way.

The term “cell culture” herein refers to the maintenance in the living state of any kind of cells, cell clusters, tissue-like structures, tissue biopsies, spheroids, organoids, or similar samples obtained or initially cultured by any method known in the art.

The term “cells” herein refers to primary, immortal or stem cells (including pluripotent or induced (in any way) pluripotent) or genetically modified cells from any type of living organism, whether archaea, prokaryote or eukaryote, and also includes viruses or other entities that need living cells to replicate.

The use of any and all examples, or exemplary language provided herein, is intended merely to better illuminate the invention and does not pose a limitation on the scope of the invention unless otherwise claimed. No language in the specification should be construed as indicating any non-claimed element as essential to the practice of the invention.

This invention includes all modifications and equivalents of the subject matter recited in the claims appended hereto as permitted by applicable law.

### BRIEF DESCRIPTION OF THE DRAWINGS

FIG. 1 schematically illustrates a perspective view of an exemplary embodiment of an incubator as disclosed herein;

FIG. 2 schematically illustrates a perspective view of the incubator of FIG. 1 with a cut-out and an enlargement;

FIGS. 3A and 3B respectively schematically illustrates a side view and a cross-sectional side view of an exemplary embodiment of a rotational drive unit receiving a cell culture chamber device, both as disclosed herein;

FIG. 4A schematically illustrates a cross-sectional side view of an exemplary embodiment of a rotational drive unit secured in an incubation chamber wall of an incubator as disclosed herein where the rotational drive unit comprises at least one light or illumination element according to some embodiments;

FIG. 4B schematically illustrates a cross-sectional side view of another exemplary embodiment of a rotational drive unit secured in an incubation chamber wall of an incubator as disclosed herein where the rotational drive unit comprises at least one light or illumination element according to other embodiments;

FIG. 5 schematically illustrates a cell culture chamber device of an incubator as disclosed herein together with a light or another illumination or visualisation signal source and an imaging, vision, or other registration or detection unit;

FIGS. 6A-6C schematically illustrate different views of an incubation chamber wall, a rotational fan, and a rotational UVC light source;

FIGS. 7A-7C schematically illustrate different views of an incubation chamber wall and a heating element;

FIG. 8 schematically illustrates a side view of an exemplary embodiment of a cell culture chamber device as disclosed herein;

FIGS. 9A-9C respectively schematically illustrates an end view of exemplary embodiments of the cell culture chamber device of FIG. 8;

FIG. 10 schematically illustrates a front view and a cross sectional side view of embodiments of a cell culture chamber device according to some embodiments and as disclosed herein comprising a circumferential gas exchanger and a circumferential humidifier FIGS. 11A-11E respectively schematically illustrates a front, a first (‘right’) side view, a first cross sectional view (AA), a second cross sectional view (CC), and a third cross sectional view (BB) of one exemplary embodiment of a cell culture chamber device as disclosed herein;

FIG. 12 schematically illustrates a perspective exploded view of the exemplary embodiment of a cell culture chamber device of FIGS. 11A-11E;

FIG. 13 schematically illustrates a perspective view of a main housing of a cell culture chamber device as disclosed herein;

FIGS. 14A and 14B schematically illustrate two perspective views of a central housing of a cell culture chamber device as disclosed herein;

FIG. 15 schematically illustrates a perspective view of a cover of a cell culture chamber device as disclosed herein; and

FIG. 16 schematically illustrates communication between a plurality of incubators and a user interface device.

### DETAILED DESCRIPTION

Various aspects and embodiments of an incubator configured to receive one or more cell culture chamber devices and of an incubator system, all as disclosed herein, will now be described with reference to the figures.

The shown figures are schematic representations for which reason the configuration of the different structures as well as their relative dimensions are intended to serve illustrative purposes only.

Some of the different components are only disclosed in relation to a single embodiment of the invention, but is meant to be included in the other embodiments without further explanation.

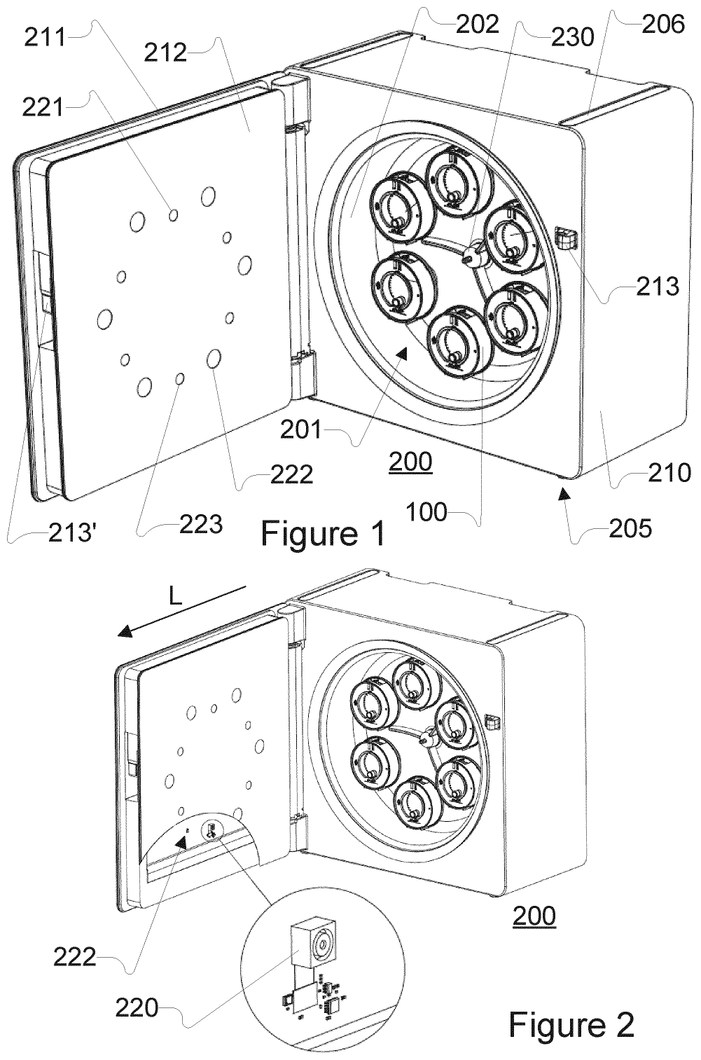

FIG. 1 schematically illustrates a perspective view of an exemplary embodiment of an incubator as disclosed herein.

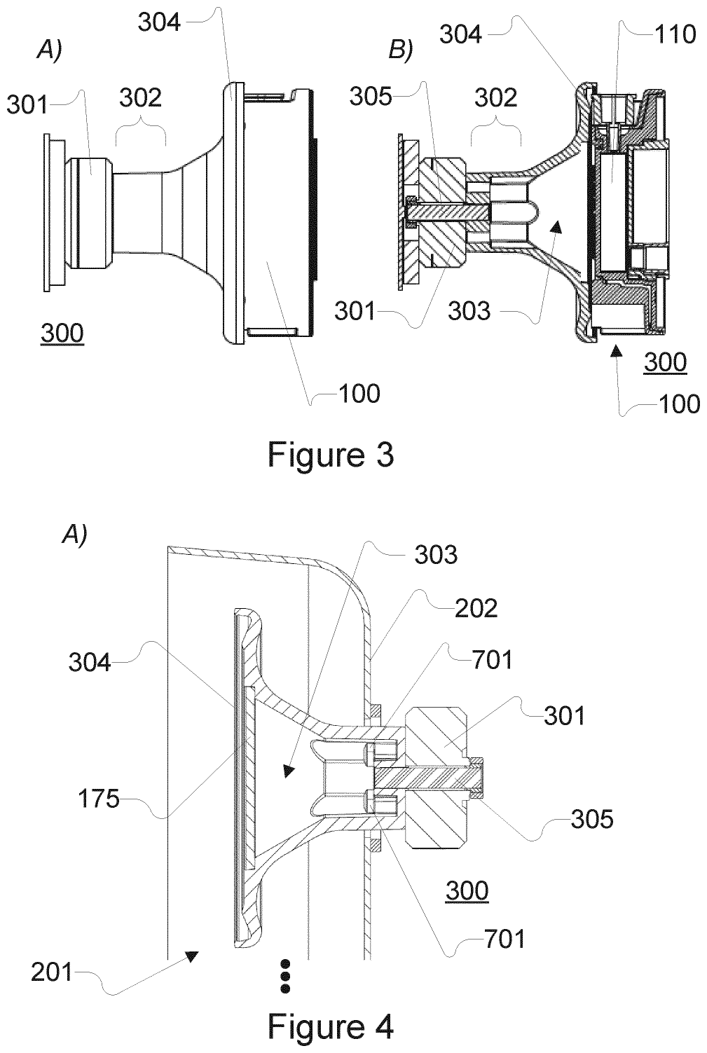

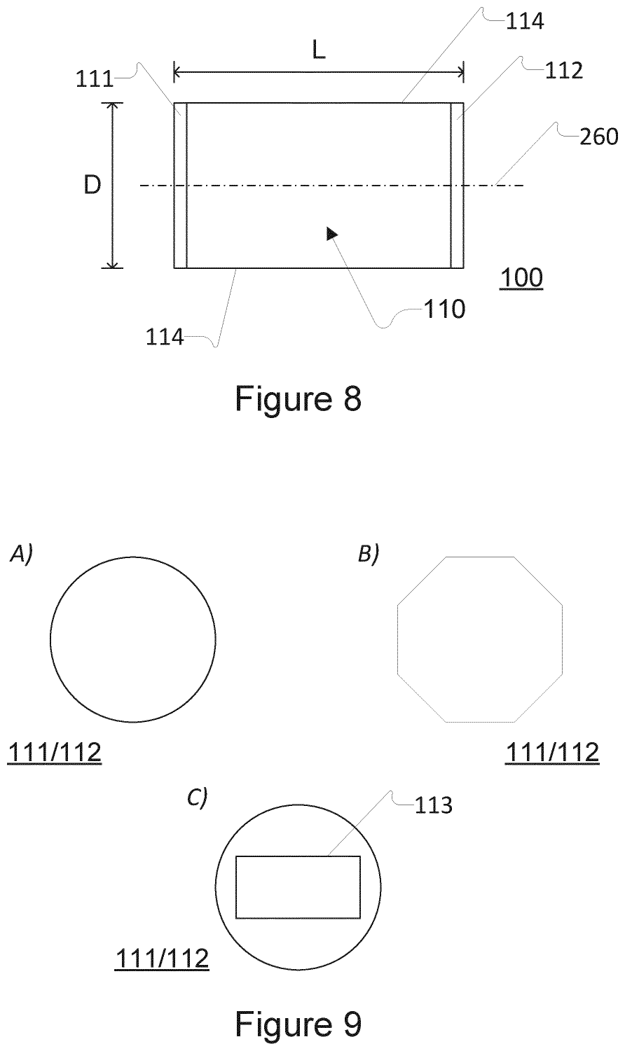

Illustrated is one embodiment of an incubator 200 configured to receive a predetermined number of cell culture chamber devices (also equally referred to herein as bioreactors) 100. The illustrated embodiment is configured to receive six, as an example, individual and independent cell culture chamber devices but other embodiments may be configured to receive different number. Each cell culture chamber device 100 to be received comprises an enclosure (also equally referred to herein as a cell chamber) (see e.g. 110 in FIG. 3B, 5, 8, 10, 11, etc.) configured to contain a cell culture media. The specific type, design, etc. of the cell culture chamber devices 100 may differ according to use and/or embodiments of the incubator 200. Various embodiments of an incubator may be adapted to receive various embodiments of cell culture chamber devices. However, expedient embodiments of cell culture chamber devices is illustrated and explained e.g. in connection with FIGS. 8-15 and otherwise disclosed herein. A single incubator 200 may e.g. be configured to receive cell culture chamber devices of different types or design.

The illustrated incubator 200 comprises a housing 210, e.g. a main housing, comprising (or defining at least in part) an incubation chamber 201 configured to contain the cell culture chamber devices 100 when received by the incubator 200, i.e. properly placed in the incubator 200 for use. The illustrated incubator 200 is configured to receive the cell culture chamber devices 100 in full within the incubation chamber 201 but other embodiments of an incubator could be configured to receive only a part of the cell culture chamber devices 100 within the incubation chamber 201 (then e.g. with another/remaining part being arranged in another part of the housing 201. The incubator 200 is configured to releasably receive the cell culture chamber devices 100. It is noted, that even if an incubator is configured to receive the predetermined number of cell culture chamber devices, here six, the incubator can receive a fewer number and operate using only these.

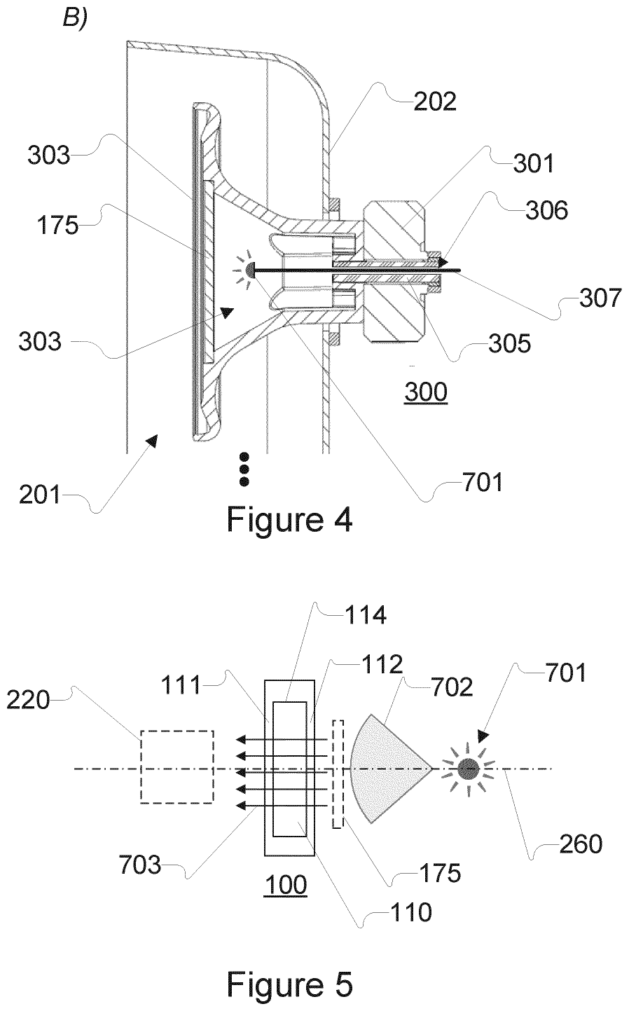

The incubator 200 further comprises a predetermined number (one or more) of rotational drive units (see e.g. 300 in FIGS. 3 and 4) configured to rotate respective received cell culture chamber devices 100 about a respective predetermined rotational axis (see e.g. 260 in FIGS. 5 and 8) of the respective cell culture chamber device 100. The rotational drive unit(s) may be configured to rotate each of the received cell culture chamber devices 100 about one, two, or three respective axes. In some embodiments, the incubator 200 comprises one rotational drive unit for each cell culture chamber device 100 the incubator 200 is capable of receiving. In at least some embodiments, the rotational drive unit(s) is/are clinostat drive unit(s). In some embodiments, back lighting of the enclosures of received cell culture chamber device(s) 100 is also provided via certain embodiments of the drive unit(s), which will be explained further in connection with FIGS. 4 and 5.

In some embodiments, a respective motor part of the rotational drive unit is located outside the incubation chamber 201 and inside housing 210 or elsewhere. This readily removes a typically heat generating component of the drive unit(s) from the incubation chamber 201 making the temperature of the environment of the incubation chamber 201 easier to control and maintain and furthermore reduces the risk of inadvertently increasing/‘spiking’ the temperature in the incubation chamber 201, which may be detrimental to the spheroids of the cell culture chamber device(s) 100 located in the chamber 201. Furthermore, by not having a motor/motor part inside the incubation chamber 201 makes it easier to keep the incubation chamber 201 clean. Exemplary embodiments of rotational drive units are shown and explained further e.g. in connection with FIGS. 3 and 4 and otherwise disclosed herein.

The incubator 200 comprises, in the illustrated embodiment, an openable and closable door, lid, or the like 211 providing access for a user to the incubation chamber 201. The door, lid, or the like 211 (when closed) together with at least one (here shown with a single) incubation chamber wall 202 of the incubation chamber 201 defines (at least in part) the incubation chamber 201.

In at least some embodiments (and as illustrated), the incubator 200 further comprises one or more locking, latching, or securing elements 213, 213′ configured so that the door or lid 211 reliably stays closed and further enables a tightly sealed off incubation chamber 201. In some further embodiments, the elements 213, 213′ are of the type providing a so-called push-to-open and push-to-close locking functionality enabling hands-free opening and closing by a user in turn reducing the risk of contamination of the incubation chamber 201. Alternatively, the elements 213, 213′ are of another type, e.g. touch-free, remotely operated, etc.

The access opening of the incubation chamber 201 is relatively large facilitating easy and convenient use for a user when taking out and inserting respective cell culture chamber devices 100.

The incubation chamber wall 202 is preferably generally ‘bowl’ shaped, i.e. generally cylindrical in a predetermined first/length direction (illustrated in FIG. 2) with no sharp corners or edges facilitating easy and efficient cleaning of the incubation chamber 201, which is significant for keeping the incubation chamber 201 an at least decontaminated environment.

According to the first aspect, the incubator 200 further comprises at least one registration and/or detection device (see e.g. 220 in FIGS. 2 and 5) configured to register and/or detect an illumination or visualisation signal (see e.g. 703 in FIG. 5) after passing, reflecting, or propagating through at least a part of respective enclosures of one or more received cell culture chamber devices 100. In some embodiments, the at least one registration and/or detection device is an imaging or vision system or device such as a camera or the like configured to register and/or detect an electromagnetic radiation, such as one or more of ultraviolet, visible, infrared, and near-infrared light. In some embodiments, the imaging or vision system or device is a more or less standard camera (e.g. of small size) configured to register (at least) visible light. Alternatively, the at least one registration and/or detection device is configured to register and/or detect sound or acoustic waves (e.g. ultrasound) or electromagnetic radiation different than visible light (e.g. x-rays).

In at least some expedient embodiments, the at least one registration and/or detection device is integrated with the incubator 200. In some further embodiments, and as shown (see also 220 in FIG. 2), the at least one registration and/or detection device is integrated into the door or lid 211 of the incubator 200 and is/are arranged behind a suitable number of windows or apertures 223 facing the incubation chamber 201.

Windows 223 are preferred over apertures to better seal the incubation chamber 201 and maintain its sterility.

In some embodiments, the incubator 200 only comprises a single or a few registration and/or detection devices arranged so that its/their field of view (FOV) readily captures a relevant part of the (relevant) cell culture chamber devices 100 when located in the incubator 200.

However, in other embodiments (and as illustrated), the incubator 200 comprises one specific and separate registration and/or detection device for each cell culture chamber device 100 that may be received by the incubator 200. In some further embodiments, each (or at least a number of) the registration and/or detection device is arranged in the door or lid 211 so that it aligns with a specific different cell culture chamber device 100 in the first or length wise direction, which often will be a horizontal direction, i.e. the registration and/or detection devices and the cell culture chamber device 100 are arranged one to one. In particular embodiments, a specific registration and/or detection device is aligned with a particular cell culture chamber device 100 so that that the particular cell culture chamber device 100 (at least its relevant part, often being at least a part of its enclosure) is within the registration or detection FOV of the specific registration and/or detection device. In some embodiments, this may entail that each registration and/or detection device is arranged so that a central axis (see e.g. 260 in FIGS. 5 and 8) of a respective enclosure 110 of a received cell culture chamber device 100 at least substantially aligns with a central axis of a field of view of registration and/or detection of a respective registration and/or detection device.

In some embodiments (with a plurality of registration and/or detection devices), the registration and/or detection devices are arranged equidistantly in a substantially circular pattern (e.g. as illustrated) in the door or lid 211 of the incubator 200.

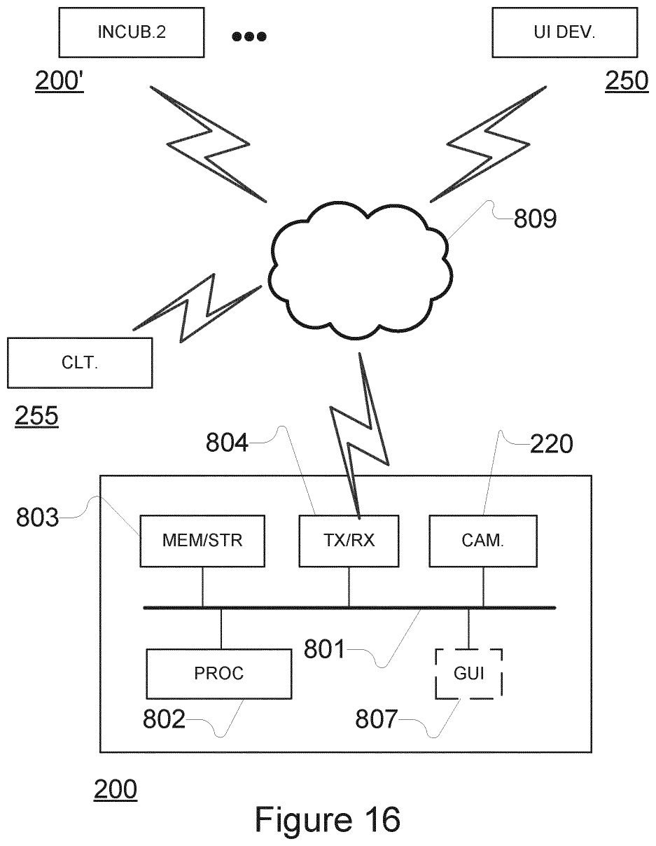

In some embodiments, the signal obtained by the registration and/or detection device(s) may be transmitted to other devices via a suitable network e.g. as illustrated and explained further in connection with FIG. 16, e.g. allowing for online monitoring, data capture, etc. of the content of the respective enclosures of the received cell culture chamber devices 100.

The at least one registration and/or detection device as disclosed herein function particularly well with cell culture chamber devices 100 respectively comprising a visible (or at least detectible) enclosure e.g. having a transparent end (at least transparent to a certain extent) (also referred to herein for some embodiments as a first end; see e.g. 111 elsewhere) facing, if necessary, the appropriate registration and/or detection device. For embodiments, comprising back-lighting or back-illumination, the at least one registration and/or detection device as disclosed herein also function particularly well with cell culture chamber devices 100 comprising an enclosure having another end (typically opposite the above mentioned transparent end) further/furthest away from the at least one registration and/or detection device being transparent or at least translucent. FIGS. 4A and 4B shows respective embodiments readily providing back-lighting or back-illumination.

In some embodiments, the incubator 200 further comprises one or more light sources 222 configured to illuminate at least a first end (see e.g. 111 in FIGS. 5, and 8-15), or a part or window (see e.g. 113 in FIG. 9) thereof, of respective enclosures of received cell culture chamber devices 100. In some embodiments (and as illustrated), the one or more light sources 222 is/are arranged in the door or lid 211 facing the incubation chamber 201 (and thereby any received cell culture chamber device(s) 100. In some further embodiments, the light source(s) 222 are located in the door or lid 211 behind a suitable window (also designated 222) (preferred) or aperture (less preferred). In some embodiments, the number of light sources is the same as the number of receivable cell culture chamber devices 100 but may readily be different. The light sources may e.g. comprise one or more suitable LED lights.

In some embodiments, the location for the cell culture chamber devices 100 (and at least in some embodiments thereby the location of the windows 223) are arranged equidistantly in a substantially circular pattern. Alternatively, other arrangements may be used. If present, the one or more light sources or its windows 222 may e.g. be located in a circular pattern somewhat between the windows 223.

In some embodiments, the first or inner side 212 of the door or lid 211, is or comprises a glass, ceramic, or other similar easily cleanable material surface facilitating maintaining a sterile environment inside the incubator 200, i.e. within incubation chamber 201. The first or inner side or surface 212 of the door or lid 211 may, as mentioned, comprise one or more windows, etc. 223 for the registration and/or detection device(s) and/or one or more windows, etc. 222 for any additional light source(s). The windows 223 may e.g. be substantially transparent, while the windows 222 may e.g. be substantially transparent or e.g. be substantially translucent e.g. in order to diffuse the light of the light sources.

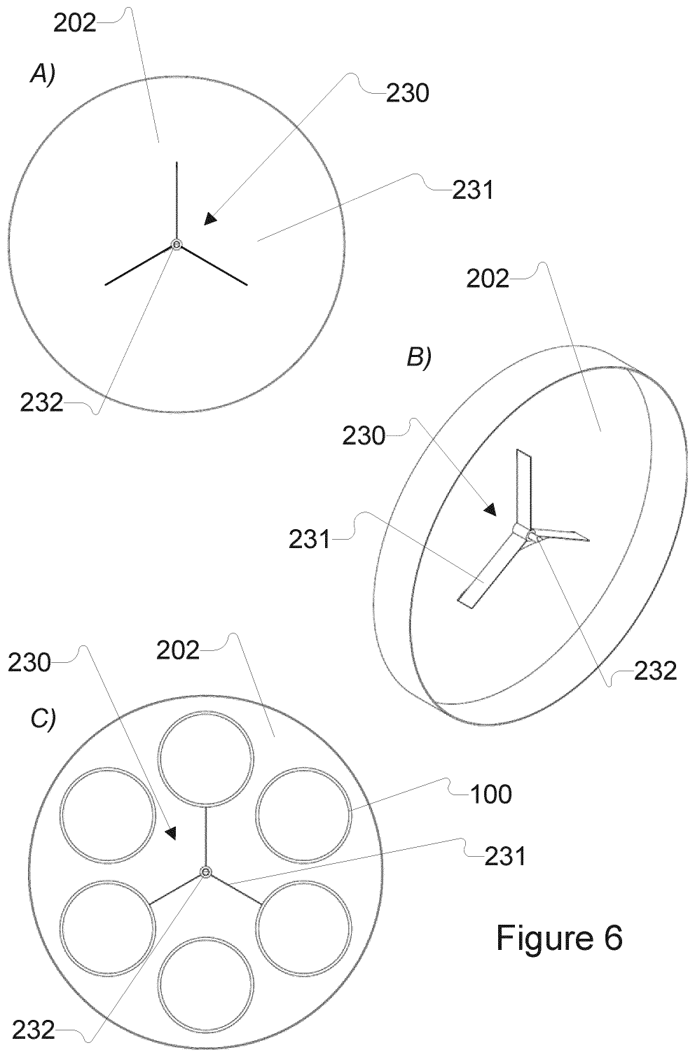

In some embodiments, the incubator 200 further comprises a fan or ventilation unit 230 arranged in the incubation chamber 201 and being configured to cause an air flow inside the incubation chamber 201 in response to a control signal. In some embodiments (and as illustrated), the fan or ventilation unit 230 is a rotating fan or ventilation unit 230. The presence of a fan or ventilation unit 230 in the incubation chamber 201 (or a fan or ventilation unit arranged elsewhere in the housing 210 but still causing an air flow in the incubation chamber 201) promotes a uniform environment within the incubation chamber 201 that typically is advantageous in relation to provide uniform humidity, uniform temperature, etc. that may be critical parameters to control in connection with the use of the incubator 200. In some embodiments (and as illustrated), the fan or ventilation unit 230 is located generally centrally in the incubation chamber 201, which also promotes the efficiency of the fan or ventilation unit 230, in particular in combination with a circular incubation chamber 201. For embodiments where the cell culture chamber devices 100 are arranged in a substantially circular pattern in the incubation chamber 201, central space is readily provided for such a central fan or ventilation unit 230 enabling a compact design. In some embodiments (and as illustrated), the central fan or ventilation unit 230 is also located ‘behind’ the cell culture chamber devices 100, i.e. between the cell culture chamber devices 100 and the bottom or wall 202 of the incubation chamber 201 (see e.g. also FIG. 6). It is to be understood, that the single central fan or ventilation unit 230 may be replaced by two or more, e.g. smaller, fan or ventilation units.

In some embodiments, the incubator 200 further comprises an arrangement for exposing the incubation chamber 201 with UVC light to decontaminate it. Use of this (at least if the UVC is intended to be activated while any cell culture chamber devices 100 are still present in the incubation chamber 201), typically requires that the cell culture chamber devices 100, or at least their enclosures, comprises UVC opaque materials or otherwise are shielded against UVC light, to shield the content inside the enclosures from exposure to the UVC light. In some embodiments, the arrangement is a UVC light source. Alternatively (not requiring the cell culture chamber devices 100 and/or their enclosures (for cell culture) to be UVC shielded), the UVC light is (to be) activated, e.g. as part of a UVC decontamination program or mode, only when the incubation chamber 201 is empty of cell culture chamber devices 100.

In some embodiments (and as illustrated), the UVC light source or similar (see e.g. 232 in FIG. 6) is integrated with a rotating fan or ventilation unit 230 providing a very compact and efficient design. Furthermore, the UVC light is thereby rotated together with the fan or ventilation unit 230 thereby sweeping the incubation chamber 201 increasing the effect of the UVC light. In some embodiments, the UVC light is asymmetrical, i.e. emits UVC light in (at least) two different, e.g. opposed, directions working particularly well in connection with rotation. An embodiment of an integrated rotating fan or ventilation and asymmetric UVC light source is shown and further explained in connection with FIG. 6.

It is noted that some embodiments, comprise only an UVC arrangement, e.g. a rotating UVC light, but no rotating fan or ventilation unit 230, no fan or ventilation unit 230 at all, or a fan or ventilation unit in another design and/or location.

In some embodiments (with UVC decontamination), at least a part of an inner surface of the incubation chamber 201 (e.g. at least a part of the incubation chamber wall 202 and/or the first or inner side or surface 212 of the door or lid 211) comprises a UVC reflecting material or coating thereby increasing the effect of the UVC light.

In some embodiments, the housing 210 is stackable in the sense that one incubator 200 as disclosed herein reliably and stably may be stacked on top of another incubator as disclosed herein. In some further embodiments, the housing 210 comprises feet, ridges, or similar 205 located underneath or at a first side of the housing 210 and one or more cavities, openings, slits, etc. 206 (mating with the feet, ridges, etc. 205) located on top or at a second side of the housing 210. In this way, several incubators 200 may be arranged compactly while allowing for incubation functionality for an even greater number of cell culture chamber devices 100 than supported by a single incubator 200. In some embodiments, the housing (and including the door or lid 211 when closed) is generally box-shaped. Accordingly, it is possible to build up/stack a number of incubator 200 in an array or the like.

FIG. 2 schematically illustrates a perspective view of the incubator of FIG. 1 with a cut-out and an enlargement.

Illustrated is the incubator 200 of FIG. 1 where an enlarged cut-out more clearly illustrates a registration and/or detection device 220 here in the form of a camera unit secured to and electrically connected to a PCB or another electrical circuit embedded in the door or lid 211. FIG. 2 also illustrates an optional light source 222 for front-lighting of at least one cell culture chamber device 100 and more specifically for front-lighting of a respective enclosure (see e.g. 110 in FIG. 3B, 5, 8, 10, 11, etc.) of at least one cell culture chamber device 100 located in the incubation chamber 201 of the incubator 200. As mentioned, the door or lid 211, in the specific shown embodiments, comprises one such camera unit 220 for each cell culture chamber device 100 that can be received by the illustrated incubator 200 where the camera units 220 respectively is aligned with a particular cell culture chamber device 100 when the door or lid 211 is closed.

FIGS. 3A and 3B respectively schematically illustrates a side view and a cross-sectional side view of an exemplary embodiment of a rotational drive unit receiving a cell culture chamber device, both as disclosed herein.

Illustrated in FIGS. 3A and 3B is an embodiment of a rotational drive unit 300 shown together with an embodiment of cell culture chamber device 100 being received by the rotational drive unit 300 (henceforth and herein equally referred to simply as drive unit), both as disclosed herein.

At least some embodiments of an incubator (see e.g. 200 in FIGS. 1 and 2) as disclosed herein comprises a number of such drive units 300, one drive unit 300 for each cell culture chamber device 100 that the incubator is designed to receive.

In the particular shown embodiment, the drive unit 300 comprising an engaging part or portion 304 configured to releasably attach the cell culture chamber device 100 to the drive unit 300 via respective releasable securing elements e.g. snap fit, friction fit, or the like elements).

The securing elements may fit only a particular type of cell culture chamber device 100 or alternatively a number of different types of cell culture chamber devices 100. The drive unit may also be able to attach with a range of different adaptors that in turn attach with a different type of cell culture chamber device 100.

The illustrated drive unit 300 only accommodate a single cell culture chamber device 100 but in alternative embodiments, a drive unit 300 might accommodate e.g. two (i.e. a dual drive unit), three, or another number of cell culture chamber devices 100.

In at least some embodiments, the drive unit 300 is configured for clinostat rotation or for rotation negating or supplementing, at least to a certain extent, the effects of gravitational pull on content in the cell culture chamber device or more specifically the content in the enclosure of an attached cell culture chamber device 100. In at least some embodiments, the drive unit 300 is able to rotate both clockwise and anticlockwise.

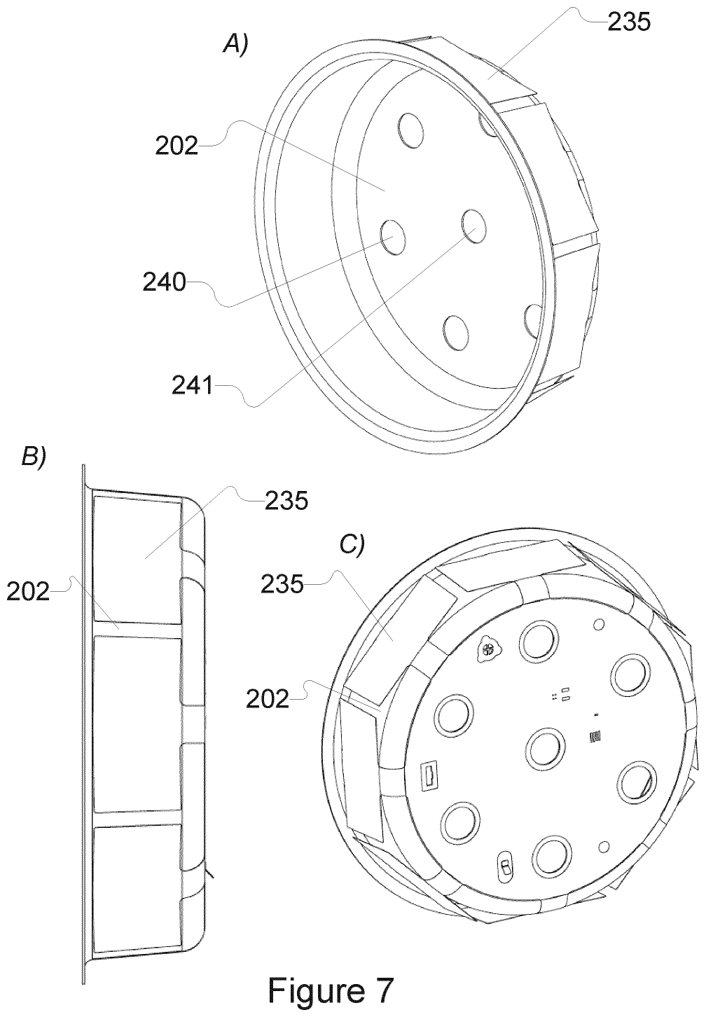

The specifically illustrated embodiment comprises a motor part comprising a motor 301 rotating a connected shaft or similar 305 in turn being connected with the engaging part or portion 304. Further indicated is a smaller/narrower section 302, i.e. with reduced diameter, which is the section that is to fit in an opening (see e.g. 240 in FIG. 7) of an incubation chamber wall or the like (see e.g. 202 in FIGS. 1, 4, 6, and 7) forming a ‘back’ or ‘end’ wall of an incubation chamber (see e.g. 201 in FIGS. 1, 4, etc.). This readily provides the motor/motor part 301 to be outside the incubation chamber (and typically inside another part of the incubator housing), which thereby removes a variable heat source from within the incubation chamber.

In alternative embodiments, another type of drive (than a rotational motor and a shaft or similar), e.g. a magnetic rotational drive or another type, may be used.

As can be seen in FIG. 3B, the drive unit 300 comprises a hollow cone- or trumpet-shaped part defining a cavity 303 and connecting the smaller section 302 with the engaging part or portion 304.

FIG. 4A schematically illustrates a cross-sectional side view of an exemplary embodiment of a rotational drive unit secured in an incubation chamber wall of an incubator as disclosed herein where the rotational drive unit comprises at least one light or illumination element according to some embodiments.

Illustrated is a drive unit 300 more or less corresponding to the one of FIG. 3 and as otherwise disclosed herein, where the drive unit 300 comprises a motor 301 rotating a connected shaft or similar 305 in turn being secured to the engaging part or portion 304 thus in turn rotating this (together with an attached cell culture chamber device). Further illustrated is (part of) an incubation chamber wall 202 defining (in part) an incubation chamber 201 (e.g. together with a door or lid or other). The incubation chamber wall 202 comprises an opening (see e.g. also 240 in FIG. 7) receiving (in an assembled state of the drive unit 300) a part (e.g. a smaller/narrower section, see e.g. also 302 in FIG. 3) of the drive unit 300. Accordingly, the drive unit 300 has the engaging part or portion 304 in the incubation chamber 201 and its motor part/motor 301 outside the incubation chamber 201. The drive unit 300 may e.g. be assembled with parts from different sides of the opening.

As mentioned, the drive unit comprises a cavity 303. According to some embodiments and as shown, the drive unit 300 further comprises at least one light source 701 (or alternatively other light or illumination element) so that light is emitted in the cavity 303 of the drive unit 300, e.g. or preferably in a direction towards the enclosure or cell culture chamber device. Accordingly, ‘back’-lighting is provided to ‘back’-light a received cell culture chamber device (see e.g. 100 elsewhere) or more particularly to ‘back’-light an enclosure (see e.g. 110 elsewhere) of a received cell culture chamber device. In some embodiments, the at least one light source 701 is configured to illuminate at least a second end (see e.g. 112 in FIGS. 5 and 8-15), or a part or window (see e.g. 113 in FIG. 9) thereof, of an enclosure of a cell culture chamber device received by the drive unit 300 in a substantially uniform manner.

Back-lighting, or more generally back-illumination, e.g. in addition to front-lighting or as an alternative, enables for much better capture (registration and/or detection) of the content of the enclosure by a registration and/or detection device (see e.g. 220 elsewhere) here in the form of a camera unit (when light is provided). This is explained further in connection with FIG. 5.

In some embodiments, the drive unit 300 comprises a predetermined number of light sources 701, in the illustrated embodiment the drive unit 300 comprises four light sources 701 (where only two are visible in the cross-sectional view). In alternative embodiments, the number of light sources 701 may e.g. be one, two, three, or another number.

In the illustrated embodiments, the four light sources 701 are offset from a centre axis or rotational axis (see e.g. 260 in FIG. 5) of the enclosure or cell culture chamber device, which may provide simple access to content of the enclosure.

The light source(s) 701 may e.g. be LED light source(s) or any other suitable light source.

If the drive unit 300 comprises a plurality of light sources, they may e.g. be of different types, e.g. emitting different wavelengths. As an example, if the drive unit comprises four LED light sources 701 then one may e.g. be emitting UV light, another visible light, a third near IR, and the fourth IR. The drive unit 300 may e.g. also comprise light sources with more than one of a same type (e.g. two of one type and one of another type and so on).

In some embodiments, the drive unit 300 may optionally comprise a light diffusor 175 may optionally be arranged in a light propagation path of light 702 from the light source 701 to or towards the enclosure 110, and in particular for the shown embodiment, be arranged before or adjacent to the second end 112.

In some embodiments (and as shown), the drive unit 300 further comprises an optional light diffusor 175 (see e.g. also FIGS. 4B and 5) arranged in light propagation path(s) between the light source(s) 701 and an enclosure of a received cell culture chamber device. As illustrated, the light diffusor 175 may e.g. be located in the cavity 303 adjacent to or at least near a received cell culture chamber device (e.g. adjacent to or near a second end of an enclosure). The light diffusor 175 will provide a more uniform lighting towards the enclosure and may therefore increase the quality of the backlighting and thereby the detection and/or registration signal of the registration and/or detection device(s).

A diffusor 175 is advantageous also in situations with several light sources of different types.

The back-lighting arrangement according to FIG. 4A provides a very compact and expedient way of enabling back-lighting for an enclosure/a cell culture chamber device in an incubator.

Alternatively, another (one or more) illumination or visualisation signal source(s) is/are used instead of light sources being configured to emit another type of illumination or visualisation signal, e.g. through a second end, or part or window thereof, into a respective enclosure, wherein the at least one registration and/or detection device is configured to capture at least a part of the other type of illumination or visualisation signal transmitted through a first end, or part or window thereof, to outside the enclosure. This other type of registration and/or detection device(s) may e.g. be configured for registration of sound or acoustic waves (e.g. ultrasound) or for registration of electromagnetic radiation different than light (e.g. x-rays). In such alternative embodiments, the diffusor 175 (if present) is not an optical diffusor but a diffusor with respect to another type of illumination or visualisation signal, e.g. an acoustic diffusor or a diffusor for electromagnetic radiation different than light.

FIG. 4B schematically illustrates a cross-sectional side view of another exemplary embodiment of a rotational drive unit secured in an incubation chamber wall of an incubator as disclosed herein where the rotational drive unit comprises at least one light or illumination element according to other embodiments.

Illustrated is a drive unit 300 more or less corresponding to the one of FIG. 4A and as otherwise disclosed herein but where back lighting is provided in another way. In the embodiment of FIG. 4B, the rotational shaft 305 the drive unit 300 comprises a, e.g. central, through-going (in the first/length direction) duct, channel, or the like 306 receiving a light guide, light rod or other light or illumination element 307 terminating in the cavity 303 and accordingly enabling back-light.

FIG. 5 schematically illustrates a cell culture chamber device of an incubator as disclosed herein together with a light or another illumination or visualisation signal source and an imaging, vision, or other registration or detection unit.

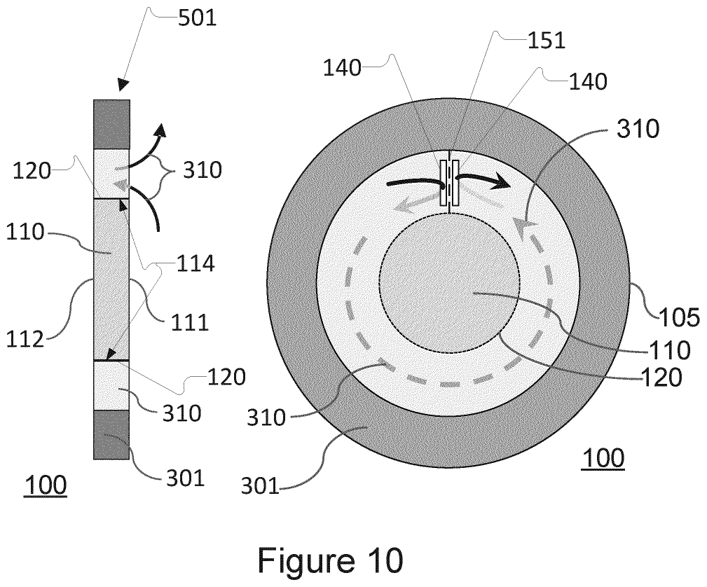

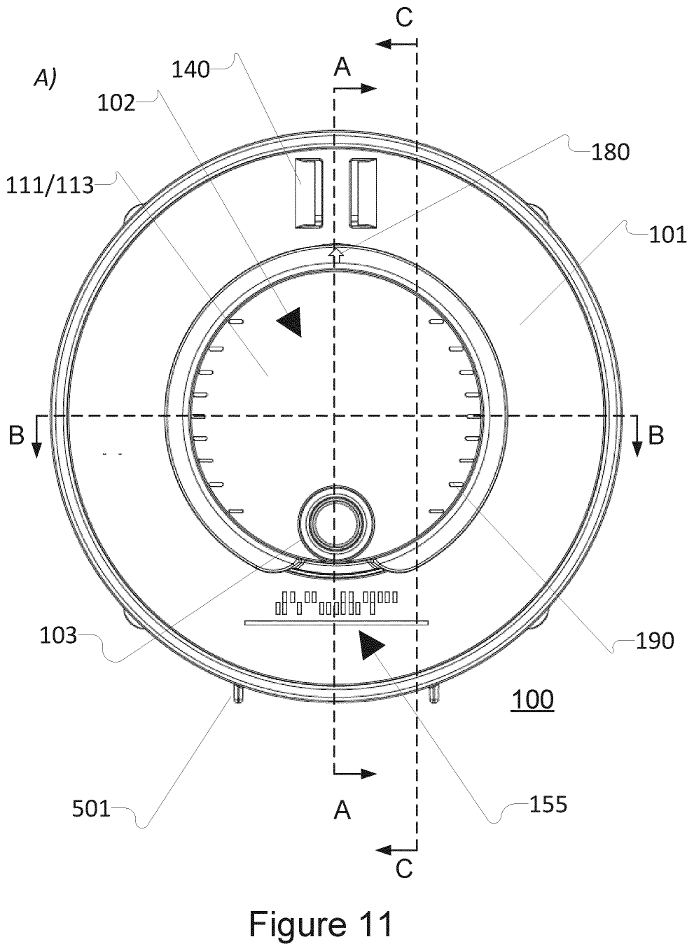

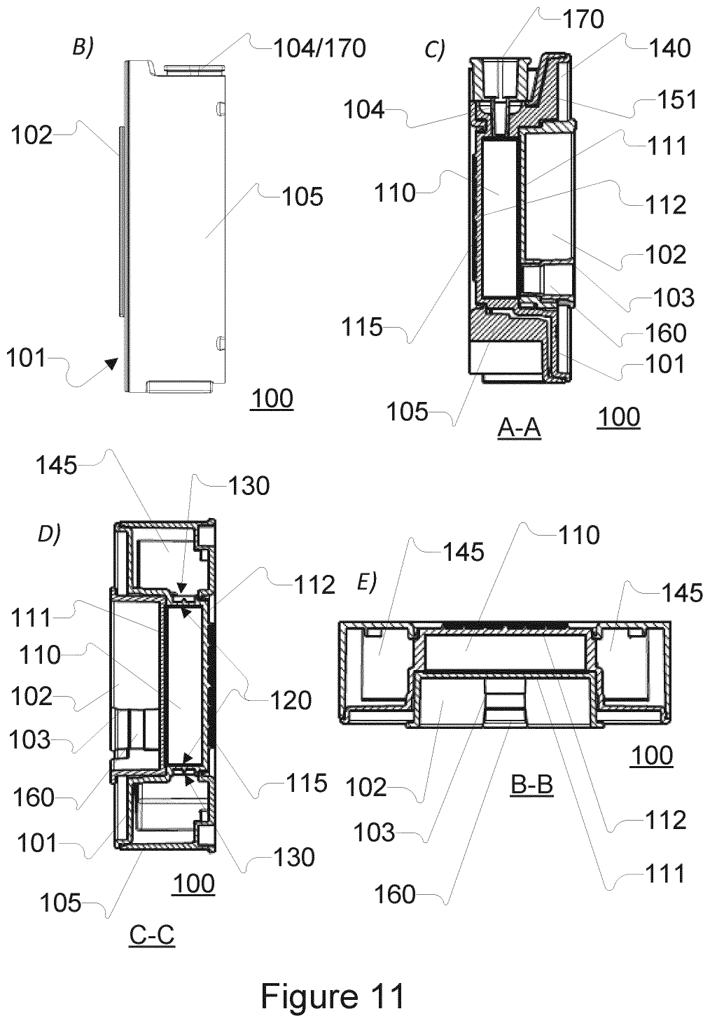

Illustrated is a cell culture chamber device 100 for the growing of cell cultures and tissues of an incubator (see e.g. 200 elsewhere) as disclosed herein. The cell culture chamber 100 comprises a first end 111, a second end 112, at least one connecting (e.g. circumferential) wall, part, segment, or the like 114 connecting the first and the second ends 111, 112 and together defining an enclosure 110 of the cell culture chamber device 100 as disclosed herein. The cell culture chamber device 100 may e.g. be substantially cylindrical, e.g. as illustrated in FIGS. 1-3, 8, and 9-15. Further illustrated is a central or rotational axis 260 that may coincide with a central axis of the enclosure 110 and/or of the cell culture chamber device 100. The cell culture chamber device 100 may also comprise a circumferential gas exchanger comprising a circumferential gas permeable membrane (not shown; see e.g. 140, 151, 310, and 120 in FIGS. 10-15). The cell culture chamber device 100 may e.g. in addition also comprise a circumferential humidifier (not shown; see e.g. 301 in FIG. 10).

In at least some embodiments and as shown, the incubator comprises (or is connected to) an imaging or vision system or device 220, such as a camera or the like, and at least one light source 701 configured to emit light 702 passing into, through, and out again of the enclosure 110 (and thereby its content), where the imaging or vision system or device 220 (see e.g. also FIG. 2) is configured to capture at least a part of light transmitted through and out of the enclosure 110 e.g. as an image or a video. The incubator may comprise (or be connected) to a plurality of imaging or vision systems or devices and/or a plurality of light sources. The light source(s) 701 may e.g. be LED light source(s) or any other suitable light sources.

The light source(s) 701 emit(s), at least in some embodiments, natural or artificial light or a combination thereof, typically or preferably visible light having a wavelength of about 400 to about 700 nanometres or at least a sub-range thereof. Alternatively, the light source(s) 701 could e.g. be outside the visible part of the electromagnetic spectrum, e.g. infrared or near-infrared light respectively having a wavelength of about 700 nanometres to about 1 millimetre or about 900 nanometres to about 2500 nanometres or ultraviolet e.g. having a wavelength of about 300 to about 400 nanometers. The light source(s) 701 may e.g. be LED light source(s).

In some embodiments and as shown, the light source 701 is located on the side of the enclosure 100 being closest to the second end 112, e.g. on or substantially on the axis 260 where the imaging or vision system or device 220 is located generally opposite to the light source 701, i.e. on the side of the enclosure 100 being closest to the first end 111. The light source 701 may e.g. be as shown and explained respectively in connection with FIGS. 4A and 4B. The imaging or vision system or device 220 may be located on the axis but does not necessarily need to be, as long as the exiting light is in its field of view. In further embodiments, the first end 111 is transparent and the second end 112 is transparent or translucent. A light diffusor 175 may optionally be arranged in a light propagation path of light 702 from the light source 701 to or towards the enclosure 110, and in particular for the shown embodiment, be arranged before or adjacent to the second end 112. If the second end 112 is translucent and/or a light diffusor 175 is present, a more uniform lighting 703 will propagate through the enclosure and outside it to be registered or observed automatically by the imaging or vision system or device 220 and/or manually by a user.

The light source 701 readily provides back-lighting of the content of the enclosure 110. In at least some embodiments, the light source 701 may be provided in a drive unit (see 300 elsewhere) e.g. as illustrated and explained in connection with FIG. 4. Instead of or in addition to back-lighting, the incubator may also comprise front-lighting as disclosed herein (see e.g. 222 in FIGS. 1 and 2). Back-lighting as disclosed herein generally improves contrast, e.g. for better seeing and/or counting whole cell structures, whereas front-lighting as disclosed herein generally increase the perceivable or registrable detail level, e.g. allowing for better seeing or registering details of the cell structure.

In alternative embodiments, the connecting wall(s), etc. 114 is transparent or translucent instead of (or in addition to) the second end 112 where the light source 701 then may be arranged adjacent or at least sufficiently close to the transparent or translucent connecting wall 114 to enable sufficient light to propagate through and out of the enclosure 110. This will not be as optimal as having the light source 701 arranged opposite the first end 111 with the enclosure 110 arranged in-between (and a transparent or translucent second end 112), but may for some uses or designs be sufficient.

In some further alternative embodiments, the light source is integrated into the second end 112 (for embodiments with a transparent/translucent second end) or integrated into the connecting wall(s) 114 (for embodiments with transparent/translucent connecting wall(s) 114).

In some additionally alternative embodiments, the second end 112 or the connecting wall(s) 114 is/are or comprises a fluorescent element, e.g. a fluorescent end 112 or a fluorescent connecting wall 114.

The fluorescent element may e.g. be an IR or a NIR induced fluorescent element or any other suitable fluorescent light source or element.

The light source(s) 701 of one or more connecting wall(s) 114 may emit light directed at or towards the first end 111.

As mentioned, the second end 112 and/or the connecting wall(s) 114 may comprise a transparent or transparent/translucent window (see e.g. 113 in FIG. 9 and elsewhere).

In some embodiments, the light source(s) 701 may be arranged inside the enclosure 110 e.g. adjacent to the transparent/translucent second end 112 (for relevant embodiments) or adjacent to the transparent/translucent connecting wall(s) 114 (for relevant embodiments).

In some alternative embodiments, at least some of the illustrated light source(s) 701 is/are replaced by one or more other illumination or visualisation signal sources 701, e.g. one or more acoustic transducers configured to emit acoustic waves, e.g. ultrasound, or one or more emitters configured to emit electromagnetic radiation other than light, e.g. infrared or x-rays. Furthermore, the imaging or vision system or device 220 is replaced by another registration or detection system or device configured to register the other illumination or visualisation signal. The other registration or detection system or device 220 may e.g. be configured for registration of sound or acoustic waves (e.g. ultrasound) or for registration of electromagnetic radiation different than light (e.g. infrared, x-rays).

In alternative embodiments, the diffusor 175 is not an optical diffusor but a diffusor 175 with respect to another type of illumination or visualisation signal, e.g. an acoustic diffusor or a diffusor for electromagnetic radiation different than light.

In some other alternative embodiments, the diffusor 175 (as disclosed herein) is replaced by a suitable reflector, e.g. a parabolic reflector, e.g. for use with front-lighting embodiments (or front-application of another type of illumination or visualisation signal) either in addition to or as an alternative to back-lighting or back-emission of another illumination or visualisation signal.

FIGS. 6A-6C schematically illustrate different views of an incubation chamber wall, a rotational fan, and a rotational UVC light source.

Illustrated are some embodiments of an integrated UVC light source or similar 232 and fan or ventilation unit 230 where both 230, 232 are configured to rotate (as one unit). The integrated UVC light source 232 and fan or ventilation unit 230 is illustrated as being arranged centrally in an incubation chamber wall 202 (at least in part) defining an incubation chamber (see e.g. 201 elsewhere).

FIG. 6A illustrates a front view and FIG. 6B illustrates a perspective view of the incubation chamber wall 202 while FIG. 6C illustrates a front view corresponding to that of FIG. 6A but where a number, here six as an example, of cell culture chamber devices 100 are received as disclosed herein.

The illustrated rotating fan or ventilation unit 230 comprises a number (here three as an example) of blades, propellers, or the like 231 configured to cause an airflow within the incubation chamber when being rotated.

The shape of the blades, propellers, etc. 231 may differ according to embodiment or implementation and FIGS. 1 and 2 illustrate differently shaped blades, etc. (being curved rather than straight) (and also shows an integrated UVC light source). Any number of and any suitably shaped blades, propellers, etc. 231 may be used.

In the illustrated embodiments, the UVC light source 232 is located centrally in the fan or ventilation unit 230 and protrudes an extent (e.g. in the first/length direction) therefrom. The illustrated UVC light source 232 is, as an example, asymmetrical and emits UVC light in (at least) two different, here opposed, directions working particularly well in connection with rotation. Accordingly, the UVC light source 232 will efficiently ‘sweep’ the inside of the incubation chamber thereby decontaminating it (requiring, as mentioned, that the cell culture enclosures of received cell culture chamber devices 100 are shielded from UVC light or that the UVC light only is activated when no cell culture chamber device(s) is/are present in the incubation chamber). Additionally, such UVC illumination will also be fairly uniformly distributed in the incubation chamber and if the incubation chamber comprises a UVC reflecting material or coating UVC light will better reach regions of the incubation chamber not directly irradiated.

It is noted that some embodiments of an incubator as disclosed herein may comprise only an UVC arrangement, e.g. a rotating UVC light 232, but no rotating fan or ventilation unit 230, may comprise no fan or ventilation unit 230 at all, or a fan or ventilation unit in another design and/or location. Embodiments may also comprise only a fan or ventilation unit 230 and no UVC arrangement/UVC light 232.

FIGS. 7A-7C schematically illustrate different views of an incubation chamber wall and a heating element.

Illustrated respectively is a perspective view (in A), a first side view (in B), and another perspective view (in C) from a different angle than in FIG. 7A illustrating an embodiment of an incubation chamber wall 202 (at least in part defining an incubation chamber) as disclosed herein comprising a number, here six as an example, of openings 240 arranged equidistantly in a substantially circular pattern and with an additional, e.g. a seventh, single opening 240 arranged in the centre of the circular pattern. The openings 240 in the circular pattern are for receiving respective drive units (see e.g. 300 elsewhere) e.g. as illustrated and explained in connection with FIGS. 4A and 4B and the central opening 241 is for receiving an UVC light source (see e.g. 232 elsewhere) and/or an fan or ventilation unit (see 230 elsewhere) e.g. integrated and rotating as explained in connection with FIG. 6.

Further illustrated is a heating element 235 for controllably heating the inside of the incubation chamber. The heating element 235 may take many suitable shapes and forms. In the illustrated and corresponding embodiments, the heating element 235 comprises a back or main part substantially matching an e.g. planar shape of the incubation chamber wall 202 that forms a ‘back’ or ‘end’ wall of the incubation chamber as well as a number of ‘arms’, ‘flaps, or ‘tongues’ extending to the side/sides of the incubation chamber wall 202 thereby effectively increasing the area of the incubation chamber wall 202 that may be directly exposed to heating in turn increasing the possible heating speed and homogeneity of the inside of the incubation chamber. It is noted that the shape of the main part of the arms, etc. may differ from the illustrated generally square shape.

An incubator as disclosed herein may comprise a temperature sensor configured to registering (enabling monitoring) of a temperature inside the incubation chamber enabling control of the temperature.

The heating element 235 may be any suitable heating element, preferably a relatively flat or thin element, such as a heating foil, grid, etc. as generally known. The heating element may e.g. be self-adhesive or be secured to the incubation chamber wall 202 in another suitable way. The illustrated heating element 235 is located on the exterior of the incubation chamber wall 202, i.e. on the surface opposite the surface inside the incubation chamber.

It is noted, that the arms, etc. of the heating element 235 in the Figure is not illustrated in their final state, i.e. they are not illustrated as being closely wrapped around the incubation chamber wall 202.

The heating element 235 together with a fan or ventilation unit (see e.g. 230 in FIG. 6) causing an airflow inside the incubation chamber enables a very uniform heating distribution within the incubation chamber, which is significant in order to effectively being able to control the temperature.

FIG. 8 schematically illustrates a side view of an exemplary embodiment of a cell culture chamber device as disclosed herein.

Illustrated is a cell culture chamber device 100 for the growing of cell cultures and tissues comprising an enclosure 110 configured to contain a cell culture media and comprising a first end 111 and a second end 112 where the first and the second ends 111, 112 at least in part defines the enclosure 110. In the shown embodiment, the first end and the second ends 111, 112 together with one or more (e.g. side or lengthwise) connecting walls, parts, segments, or the like 114 define the enclosure 110 and the cell culture chamber device 100 has—as an example—an overall substantially cylindrical shape with the first and the second ends 111, 112 respectively forming the circular bases of the cylinder (see also FIGS. 9A, 9B, 11, etc.). In case of an overall substantially cylindrical shape, only a single (circumferential) wall, part, segment, etc. is present connecting the first and second ends 111, 112. In some embodiments, at least one or more parts, but e.g. all, of the connecting wall, etc. 114 of the enclosure 110 is constituted by a circumferential gas permeable membrane (see e.g. 120 in FIGS. 10, 11A, and 12). It is further noted, that the enclosure 110 does not need to, and often will not, fill the entire extent of the cell culture chamber device 100 (see e.g. the following figures). In such embodiments, a housing and/or a number of housing parts (see e.g. 105, 101, and 102 in the following) may comprise the enclosure 110 (and thereby the ends 111, 112 and the one or more walls connecting the ends 111, 112).

Further illustrated is a central axis 260 extending between the first and second ends 111, 112. In at least some embodiments (and as illustrated), the cell culture chamber device 100 is configured for rotation about the central axis 260 e.g. as generally known. In at least some embodiments, the enclosure is symmetrically located in the cell culture chamber device 100 with respect to the axis of rotation/the central axis 260.

According to the first aspect, the first end 111 or a part or window (see e.g. 113 in FIGS. 8 and 9C) thereof is substantially transparent and the second end 112 or a part or window (see e.g. 113 in FIGS. 8 and 9C) thereof is substantially transparent or is substantially translucent. Furthermore, the first end 111 or the part or window thereof is configured to be optically or otherwise aligned with the second end 112 or the part or window thereof as disclosed herein. In this way, light or another illumination or visualisation signal (see e.g. 703 in FIG. 5) received through the second end 112 (or the part or window thereof) and/or through the at least one sidewall 114 (or the part or window thereof) into the enclosure 110 is transmitted through the cell culture media and out through the first end 111 or the part or window thereof to outside the enclosure 110, and e.g. outside the cell culture chamber device 100.

In some embodiments, the cell culture chamber device 100 comprises a gas exchange circuit, element, or system (not shown; see e.g. 130, 140, 151, 310, etc. in FIGS. 10, 11, and 12; equally referred to simply as a gas exchanger herein), e.g. as disclosed herein, configured to exchange gas (e.g. or primarily oxygen and carbon dioxide) into (e.g. oxygen most often but sometimes carbon dioxide) and out (e.g. carbon dioxide most often but sometimes oxygen) of the enclosure 110. In at least some preferred embodiments, the gas exchange circuit or system is a circumferential gas exchange circuit, element, or system (see e.g. 130, 140, 151, 310, etc. elsewhere) as disclosed herein comprising a gas permeable membrane (see e.g. 120 in FIG. 10 and elsewhere). Alternatively, gas exchange can occur either through the sidewalls 114 of the enclosure 110, e.g. if the material of the lengthwise sidewalls 114 are polydimethylsiloxane (PDMS) (that may be produced so it is transparent) or similar, or through special filters mounted in one or more of the ends/walls 111, 112, 114. The cell culture chamber device 100 may comprise one or more special filters and/or gas inlets/outlets allowing for transfer or exchange of gas with the enclosure 110. As another alternative, the cell culture chamber device 100 is functionally connected to a gas exchange circuit or system being external to the cell culture chamber device 100.

In some embodiments, the cell culture chamber device 100 comprises a humidifier e.g. as disclosed herein. In at least some embodiments, the humidifier is configured to humidify the gas or air close to or in the vicinity of the gas exchanger and/or the gas or air that is provided to the enclosure 110. In at least some embodiments, the humidifier is preferably a circumferential humidifier (see e.g. 301 in FIG. 10) as disclosed herein. Alternatively, the cell culture chamber device 100 is functionally connected to a humidifier being external to the cell culture chamber device 100. As yet another alternative, the cell culture chamber device 100 is intended for use within an incubator or similar, providing a controlled humidified environment as generally known, in which case the cell culture chamber device 100 does not require a humidifier.

The presence of a humidifier system will eliminate or at least significantly reduce loss of liquid from the enclosure 110 and will greatly enhance the gas exchange between the enclosure 110 and the surrounding air or atmosphere for certain types of cell culture media. The difference is so significant that the cell culture chamber device 100 will normally be able to be used e.g. in an incubator without additional humidification. This typically also reduces the risk of infection in the incubator as its gaseous environment then does not need to be as humid/humidified.

Further illustrated is a first extent or length ‘L’ and a second extent or height or (in case of e.g. a cylindrically shaped cell culture chamber device) diameter ‘D’ of the cell culture chamber device (100). The extents define, at least in part, a form-factor of the cell culture chamber device 100. In some embodiments (as illustrated), L is larger than D. However, in other embodiments (see e.g. FIGS. 1, 2, 3, 5, 10, and 12-15.) L is smaller than D, i.e. the circumferential extent is larger than the lengthwise extent. In some embodiments, the ratio between L and D is about 1:1 to about 1:10, e.g. about 1:2 to about 1:5. In further embodiments, the ratio is about 1:3 to about 1:4.

The cross sections (and/or the shapes) of the first end 111 and the second end 112 may be different from each other or be the same or similar.

FIGS. 9A to 9C illustrate differently shaped cross sections of the first and/or second ends 111, 112 according to different exemplary embodiments.

The cell culture chamber device 100 may comprise one or more conduits, inlets, or access ports (not shown; see e.g. 103, 104, 140, 160, 170, etc. in subsequent Figures) e.g. gas inlet/outlet for the humidifier, liquid inlet/outlet connected to the enclosure 110 to provide access to contained cell culture media e.g. for taking out a sample of the enclosure 110 or introducing cell culture media or another liquid into the enclosure 110.

In some embodiments, (at least one of) the ends 111, 112 is a removable cover or lid providing access to the enclosure 110 when removed.

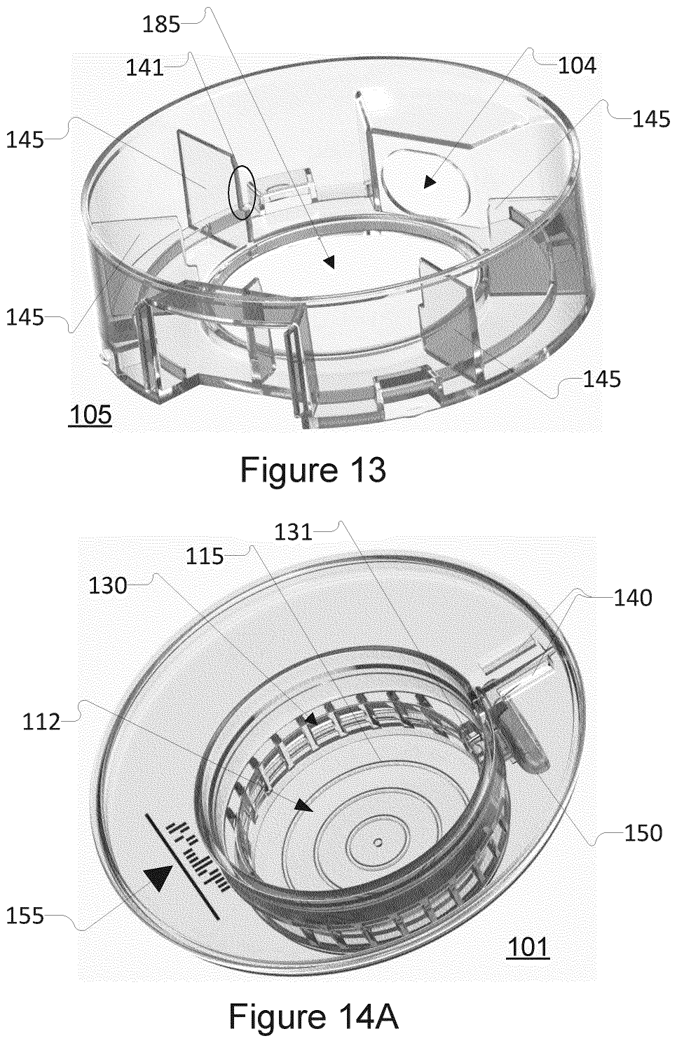

In some embodiments, all or substantially all the material of the cell culture chamber device 100 is transparent (rather than only one or both ends 111, 112) (see e.g. also FIGS. 8-10). In some further embodiments, all or substantially all the material of the cell culture chamber device 100 is transparent (including the first end 111) (see e.g. also FIGS. 13-15) except the second end 112 that is translucent.

In some embodiments one or more ports or inlet/outlets may be used to change growth media (nutrients), add (potentially biologically active) compounds, virus, bacteria, etc. and other to the content of the enclosure 110, remove spheroids from the enclosure 110, etc.

FIGS. 9A-9C respectively schematically illustrates an end view of exemplary embodiments of the cell culture chamber device of FIG. 8.

Illustrated in FIGS. 9A and 9B is respectively an end view or a cross-sectional view of one (or both) of the ends 111, 112. As can be seen, the shape is circular (e.g. for cylindrically shaped cell culture chamber devices) in FIG. 9A and is, as an example, octagonal in FIG. 9B. As mentioned, the shape of the ends (and of the cell culture chamber device) may be any suitable shape e.g. as disclosed herein.

Shown in FIG. 9C is an end view or a cross-sectional view of one (or both) of the ends 111, 112. This corresponds to FIG. 9A except that the end(s) comprises a window 113. In such embodiments, it is the window(s) that are transparent (or translucent) rather than the entire ends 111, 112. The size and location of the window(s) 113 may be any suitable size and location but preferably should readily allow for visual or other type of inspection and control of the content of the enclosure 110.

FIG. 10 schematically illustrates a front view (shown to the right in the Figure) and a cross sectional side view (shown to the left in the Figure) of embodiments of a cell culture chamber device according to some embodiments and as disclosed herein comprising a circumferential gas exchanger and a circumferential humidifier.

Illustrated (see both views) is a cell culture chamber device 100 as disclosed herein. The cell culture chamber device 100 comprises an enclosure 110 as disclosed herein defined by a first end 111, a second end 112, and at least one connecting wall 114 connecting the ends 111, 112. The enclosure 110 is e.g. comprised by a housing/a main housing 105 where the main housing 105 is cylindrical (as an example) and centrally (as an example) comprises the enclosure 110. In the shown and corresponding embodiments, the at least one connecting wall 114 is constituted by a (supported) circumferential gas permeable membrane 120 arranged along or as a circumferential part, i.e. the perimeter or part thereof, of the enclosure 110 and being configured for exchange of gases, e.g. oxygen and carbon dioxide. The circumferential gas permeable membrane 120 may e.g. be a semipermeable membrane.

Humidification of the atmosphere close to or in the vicinity of the circumferential gas permeable membrane 120 will typically reduce or avoid cell culture media evaporation and may for certain cell culture media furthermore greatly facilitate the exchange of gases through the circumferential gas permeable membrane 120. Cells produce CO₂ which in solution combines with water to form bicarbonate (which is acidic). Humidification of the atmosphere results in the outer surface of the circumferential gas permeable membrane 120 becoming humid and this facilitates the escape of CO₂ from the culture media and in doing so slow the acidification process. This process occurs in types of cell culture that do not rely on CO₂ to buffer the media (e.g. those that contain HEPES, a zwitterionic sulfonic acid buffering agent). The most widely used types of growth media rely on bicarbonate in the media and CO₂ in the atmosphere to buffer the pH of the media. Here also humidification of the outer surface of the circumferential gas permeable membrane facilitates the ‘capture’ or ‘release’ of CO₂ improving stabilisation of the pH of the medium. Humidification can be provided by the cell culture chamber device 100 being located in a humidified incubator or by a humidifier as described in the following.

The cell culture chamber device 100 comprises, as shown by the front view (to the right in the Figure), a gas exchange intake and outlet for a gas exchanger that may be any suitable intake, conduit, etc. Preferably, and as shown, the gas exchange intake and outlet is in the form of a double vent or similar 140 (see e.g. also 140 e.g. in FIGS. 11, 12, and 14A) connecting the circumferential gas exchanger with outside or ambient air or gas of the cell culture chamber device 100. In the shown embodiment, the double vent 140 is, as an example, located on a front side or front facing side (see also later Figures) or similar of a housing or main housing 105. In this particular (and corresponding embodiments), the gas exchanger comprises a gas permeable membrane 120 configured to exchange gases, e.g. oxygen and carbon dioxide, with the enclosure 110/the content of the enclosure (e.g. cell culture media). In particular, oxygen may be provided into the enclosure 110 and carbon dioxide may be removed from the enclosure 110. In the shown and corresponding embodiments, the membrane 120 constitutes the (at least one) connecting wall 114 of the enclosure 110 or one or more parts thereof.

The gas exchange intake and outlet/the double vent 140 is in fluid connection with the membrane 120 thereby connecting the membrane 120 with outside or ambient air or gas of the cell culture chamber device 100. In at least some embodiments, the double vent 140 is configured to operate according to the Coand\check{a} effect or principle. In such embodiments, a wall or other suitable barrier 151 (indicated in the Figure by a straight dashed line) is located in-between the two respective vents of the double vent 140 separating and sealing them from each other at this location, i.e. in this particular example separating and sealing them in the shortest direction between them. However, the two vents of the double vent 140 are in fluid connection with each other via another path inside the housing 105 of the cell culture chamber device 100 and are also in fluid connection with at least parts of the gas exchange membrane 120 e.g. via one or more conduits, open spaces, cavities, etc. When the cell culture chamber device 100 is rotated anticlockwise, ambient air or gas is sucked into and out of the cell culture chamber device 100 via the double vent 140 as indicated by the arrows of the front view and cross-sectional side view of FIG. 10. As can be seen, air or gas is, during anticlockwise rotation, more specifically sucked into the cell culture chamber device 100 by the left (in the front view) vent 140 as indicated by the arrow going from black to grey and expelled outside the cell culture chamber device 100 by the right (in the front view) vent 140 as indicated by the arrow going from grey to black creating an internal air flow 310 with a direction as indicated by the light grey dashed circular arrow. This is the case for anticlockwise rotation. If the cell culture chamber device 100 is rotated clockwise, the direction of the airflow 310 inside the housing 105 will reverse due to symmetry, i.e. the light grey dashed circular arrow will be clockwise and air or gas is sucked in by the right vent 140 and expelled by the left vent 140.

In this way, an effective air flow 310 is readily provided being in contact with the membrane 120 and the ambient gas or air thereby e.g. expediently adding oxygen and removing carbon dioxide to/from the membrane 120 and thereby the content of the enclosure 110.

In some further embodiments, the degree of air movement or flow 310 can be regulated by regulating the respective sizes of openings of the vents of the double vent 140 for example with a slider or small or differently sized plugs or in another suitable manner.

In some further embodiments (and as shown), the cell culture chamber device 100 optionally further comprises a circumferential humidifier or humidification or moisturising element or system (herein equally referred to as humidifier) 301 configured to humidify or moisturise air or gas at least in the vicinity of the membrane 120 (at least parts thereof). A humidifier will greatly enhance a gas exchange between the content of the enclosure 110 and the ambient air or gas and will furthermore reduce or eliminate water or liquid loss from the enclosure 110 when containing a liquid or aqueous solution. The effect is so significant that the cell culture chamber device 100 will normally be able to be used in an incubator without additional humidification. This is advantageous since it typically will reduce a risk of infection in the incubator and also enables simplification of the incubator.

In some such embodiments, the circumferential humidifier 301 comprises (or is connected to) one or more liquid or moisturising reservoirs or elements. It is advantageous if the weight distribution of the circumferential humidifier 301 is at least somewhat uniformly distributed, at least to some extent, about a central axis or a rotational axis of the cell culture chamber device 100. It is also an advantage if such one or more liquid or moisturising reservoirs or elements has, or provides, a relatively large surface area for evaporation.

There are several expedient possibilities for humidifying or moisturising air or gas at least in the vicinity of the membrane 120 (at least parts thereof).

In some embodiments, the circumferential humidifier 301 comprises an element or reservoir containing (preferably sterile) liquid water or other moisturising liquid e.g. with one or more suitable filters, outlets, further (gas permeable and particularly semipermeable) membranes, etc. interfacing the water or liquid with the air flow 310 thereby humidifying or moisturising the air flow 310. The element or reservoir may e.g. be a single circumferential unit or alternatively be several separate and distinct units (e.g. evenly distributed about the central and/or rotational axis).

In alternative embodiments, the circumferential humidifier 301 comprises one or more of a water or solute-containing material such as a gel, sponge, a particulate material (e.g. water-beads, aqua-beads, etc.) that readily provides evaporation of water or liquid and efficiently influences the air flow 310. Such solid humidifying or moisturising elements may be supported or secured in the housing 105 e.g. by or to an (open) enclosure, a wall or other support structure (e.g. 145 in the following Figures).

In case of water-beads or a gel, these may be secured, adhered, pasted, etc. to an inner wall (as mentioned e.g. or preferably uniformly about the central and/or rotational axis) of the main housing/housing 105, whereby support structures are not necessary.

For embodiments not comprising a water or liquid reservoir (e.g. water-beads, gel, etc. as mentioned above) it is possible to locate such directly in a conduit, cavity, open space, etc., comprising the air flow 310, thereby greatly increasing the humidifying or moisturising effect of the air flow 310 and enabling reduction of overall space/foot-print of the cell culture chamber device 100.

It is noted, that for embodiments without a humidifier (e.g. for use in a humidified incubator or other), the shown cell culture chamber device 100 will not comprise the illustrated circumferential humidifier 301 and may have a reduced size as a result.

FIGS. 11A-11E respectively schematically illustrates a front, a first (‘right’) side view, a first cross sectional view (AA), a second cross sectional view (CC), and a third cross sectional view (BB) of one exemplary embodiment of a cell culture chamber device as disclosed herein.

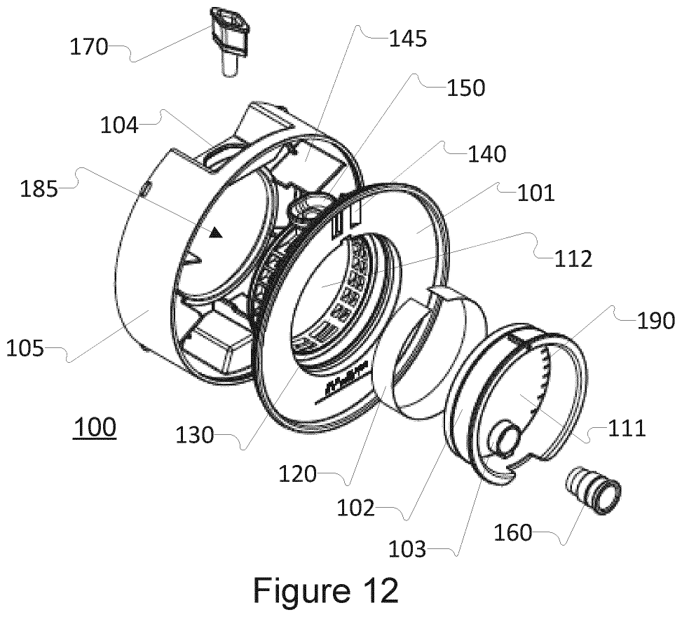

Illustrated in FIG. 11A is a front view of an exemplary preferred embodiment of a cell culture chamber device as disclosed herein. Illustrated is a front of a cell culture chamber device 100 comprising a transparent first end 111. In this particular (and corresponding embodiments) a floor, bottom, or wall of a cover 102 (see e.g. also 102 in FIGS. 11 C-E, 12, and 15) constitutes a first end 111 (or a part or window 113 thereof) of the cell culture chamber device 100. The floor or bottom of the cover 102 form an enclosure (see e.g. 110 in FIGS. 110-11E and elsewhere) as disclosed herein together with a first or central housing 101 as will be more apparent from some of the following figures. In some embodiments (and as shown in FIGS. 11C, 11D, 11E, 12, etc.), the first or central housing 101 comprises a central cavity for (e.g. or preferably releasable) receipt of at least a part of the cover 102 and more particularly (in the shown and corresponding embodiments) for receipt of the floor or bottom of a cover 102. The first or central housing 101 and the cover 102 may e.g. comprise respective releasable securing elements (such as snap fit, bayonet, friction fit, etc. elements) to releasably secure them together. Alternatively, they may be fixed non-releasably to one another or e.g. be integrally formed.

The cell culture chamber device 100 of FIGS. 11A-11E is, as an example, shaped substantially cylindrically with a circular first end.

In this particular (and corresponding embodiments), the central housing 101 additionally comprises a gas exchange circuit, element, or system in the form of a circumferential gas exchange system comprising a circumferential gas permeable membrane (not shown; see e.g. 301 and 120 in FIGS. 10, 11D, and 12). As mentioned, the floor or bottom of the cover 102 constitutes a first end 111 (or a part or window 113 thereof) of the cell culture chamber device 100.

In this particular (and corresponding embodiments), the central housing 101 furthermore comprises a circumferential humidifier (not shown) as disclosed herein and e.g. as explained in connection with FIG. 10). Some alternative embodiments of the cell culture chamber device 100 do not comprise any humidifier, e.g. for use in a humidified incubator or other.

The central housing 101 optionally comprises a gas exchange intake and outlet for a gas exchanger as disclosed herein (see e.g. 130, 140, 151, 310, etc. in FIGS. 10-15) in the form of a double vent 140 located on the front of the central housing 101. The double vent 140 has been described in more detail e.g. in connection with FIG. 10.

Further indicated are three cross-sections designated AA (shown in FIG. 11C), BB (shown in FIG. 11E), and CC (shown in FIG. 11D).

In some embodiments (and as shown), the cell culture chamber device 100 further comprises a closable and/or sealable (first) port 103 providing access for a user to the inside of the enclosure e.g. for taking out a sample from the enclosure (e.g. removing spheroids), emptying or filling the enclosure, etc. In the shown embodiment, the closable and/or sealable port 103 comprises a conduit (from the inside of the enclosure to outside the cell culture chamber device 100) and e.g. a simple plug or similar 160. The port 103 may advantageously be located on the top of the cell culture chamber device 100 as this may avoid or reduce bubble formation, e.g. by allowing for overflow.

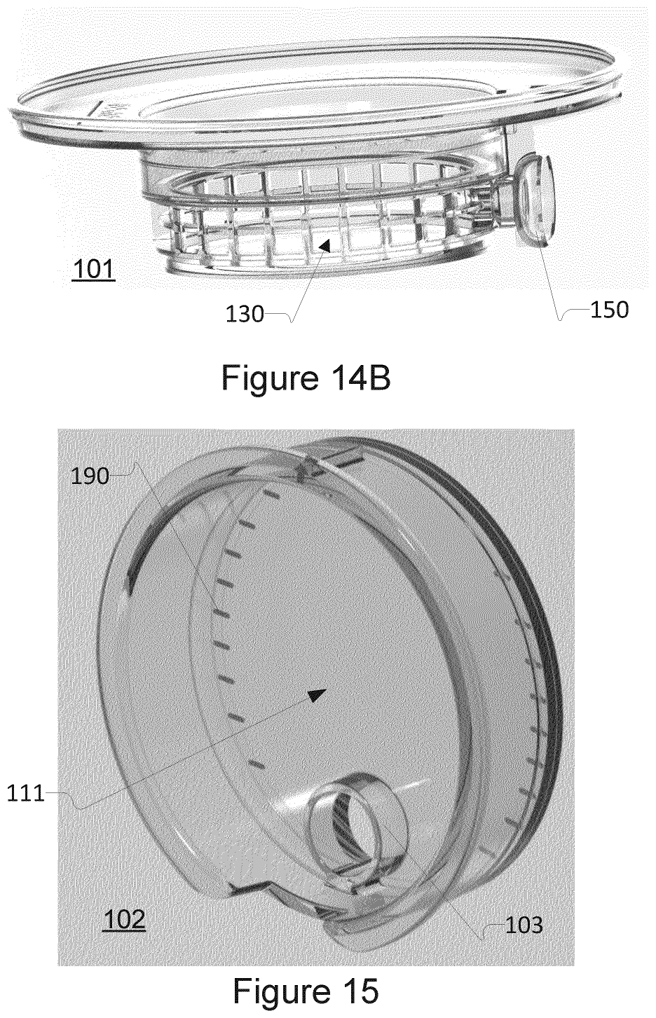

In some embodiments (and as shown), the cell culture chamber device 100 further comprises one or more fiducial and/or identification markers, here an identification code 155 and a fiducial marker 180. The identification code 155 is preferably unique to the particular cell culture chamber device 100. The fiducial marker 180 enables determination of the orientation of the cell culture chamber device 100. The fiducial and/or identification markers 155, 180 is/are preferably machine readable, e.g. by a suitable imaging or vision unit or system such as the at least one registration and/or detection device as disclosed herein (see e.g. 220 elsewhere). In some embodiments, the cell culture chamber device further comprises one or more aligning elements (e.g. location bar and slit or slot, etc.) for aligning different parts (ensuring or facilitating that a part may only be received with a proper orientation by another part) of the cell culture chamber (e.g. appropriately aligning the cover 102 with the first or central housing 101). The fiducial marker 180 may e.g. be such an aligning element (see e.g. also 131 in FIG. 14A).

Accordingly, a very compact (lengthwise) cell culture chamber device 100 is provided, in particular because of the circumferential gas exchange system and (if present) the circumferential humidifier. That the gas exchange system and (if present) the humidifier are circumferential also entails that they do not block a line of sight for any registration and/or detection device registering and/or detecting content of the enclosure of a cell culture chamber device 100.

Optionally, the transparent cover 102 comprises a number of level or fill-rate indicators 190 readily indicating an actual volume of cell culture media contained in the enclosure.

In some embodiments and as shown, the cell culture chamber device 100 further comprises one or more (here two) feet, standing elements or the like 501 enabling the cell culture chamber device 100 to stand and prevent it from rolling. This may make use of the ports, inlets, etc. easier or more reliable (see e.g. port 170 in the following).

Illustrated in FIG. 11B is a side view of the cell culture chamber device 100 of FIG. 11A seen from the side and from right to left (according to the orientation of FIG. 11A). The shown cell culture chamber device 100 comprises a main housing 105 receiving (e.g. permanently or in a fixed way) the central housing 101 in turn receiving (e.g. releasably) the cover 102. Further shown is a (second) port 104 (or rather a plug or valve thereof 170) that is in fluid connection with and provides (additional) access to the enclosure.

The ratio between a first extent/length (in the left right direction of FIG. 11B) and the second extent/height or diameter (in the up down direction of FIG. 11B) is about 1 to about 3-4 e.g. about 1 to about 3.5 but may be different, e.g. as disclosed herein, for other embodiments.

Illustrated in FIG. 11C is a first cross sectional view as given by A-A of FIG. 11A. Illustrated is the enclosure 110 defined by the transparent first end 111 (being the floor or bottom of the cover 102, the transparent or translucent second 112 being a floor or bottom of the central housing 101, and sidewalls of the cavity of the central housing 101. Further illustrated is the main housing 105 receiving the central housing 101 and the cover 102 in a very compact way.

As mentioned, the second port 104 provides access (in addition to the first port 103) to the enclosure. As explained in connection with e.g. FIG. 3, the double vent 140 connects the outside or ambient air or gas of the cell culture chamber device 100 with the circumferential gas exchange system (see e.g. 130, 140, 151, 310, etc. elsewhere).

As can be seen, the closable and/or sealable first port 103 and its conduit connects the inside of the enclosure 110 to outside the cell culture chamber device 100. The port walls are a part of the cover 102, allowing for easy access to the content of the enclosure 110. In a similar manner, access to the inside of the enclosure 110 is afforded via the second port 104 (with plug 170). The plug walls of 104/170 are a part of the central housing 101. It is noted, that the first port 103 and the second part 104 are arranged at different sides of the cell culture chamber device 100 enabling easy access to the enclosure from several different sides of the cell culture chamber device 100.

Further illustrated is the gas exchange intake and outlet in the form of a double vent 140 as already explained.

The view of FIG. 11C is a central vertical cut viewed from left to right (in the orientation according to FIG. 11A).

Illustrated in FIG. 11D is a second cross sectional view as given by C-C of FIG. 11A and viewed from right to left.

Again, the enclosure 110, the first transparent end 111, the transparent or translucent second 112, the central housing 101, the cover 102, the closable and/or sealable ports 103 and 104, and the main housing 105 are illustrated.

Further shown, is the gas permeable membrane 120 of the circumferential gas exchange system and a (part of a) grid like structure 130 of the circumferential humidifier (see e.g. 130 in FIGS. 14A and 14B).

Also illustrated is an optional wall structure element or similar 145 for holding and/or supporting a water, liquid, or moisturizing element (explained further in connection with FIG. 13) according to some embodiments of a circumferential humidifier.

In some embodiments, the cell culture chamber device 100 optionally further comprises one or more markings 115 (see also 115 in FIG. 14A)—herein as an example in the form of a number of concentric circles 115 that may give a user some fixed marks against which to see the gentle movement of the contained spheroids. The markings 115 are (as an example) arranged on the ‘outside’ of the second end 112.

The view of FIG. 11D is a vertical cut shifted off-centre to the left and viewed from right to left (in the orientation according to FIG. 11A).

Illustrated in FIG. 11E is a third cross sectional view as given by B-B of FIG. 11A.

Illustrated is the enclosure 110, the first transparent end 111, the transparent or translucent second 112, the cover 102, the closable and/or sealable port 103, and two optional wall structure elements or similar 145 for holding and/or supporting a water, liquid, or moisturizing element according to some embodiments.

The view of FIG. 11E is a horizontal central cut viewed from top to bottom (in the orientation according to FIG. 11A).

It is noted, that the cell culture chamber device 100 readily enables inspection, automatic and/or manual, of the content of the enclosure 110 from two sides (as given by the first and the second ends 111, 112) if both ends 111, 112 are transparent.

In the embodiment illustrated in FIGS. 11A-11E (and corresponding ones), the second end 112 is preferably translucent rather than transparent as this provides better (more uniform) lighting of the content of the enclosure 110 while avoiding a need for a light diffusor.

FIG. 12 schematically illustrates a perspective exploded view of the exemplary embodiment of a cell culture chamber device of FIGS. 11A-11E.

Illustrated are the elements of FIGS. 11A-11E shown in an exploded view.

FIG. 12 more clearly show the grid like structure 130 of the circumferential humidifier and the gas permeable membrane 120. In an assembled state of the cell culture chamber device 100, the gas permeable membrane 120 is located adjacent to an inside of the grid like structure 130 and the first end 111 is opposite (and optically aligned with) the second 112 and the second end 112 is aligned with an opening 185 of the main housing 105 readily enabling inspection of the content of the enclosure from that side also if the second end 112 is transparent. Further shown is a further port 150 that aligns with the first port 104 in the assembled state.

If the second end 112 is transparent, it is possible to provide lighting (‘back’-lighting) from this side through the opening 185 of the main housing 105 thereby enhancing inspection (manual or automatic) from the other/opposite side (via the first end 111).

If the second end 112 is translucent, suitable (back-)lighting may provide a more uniform illumination of the content of the enclosure further enhancing inspection (manual or automatic) from the other/opposite side (via the first end 111).

If the second end 112 is transparent (or translucent), uniform lighting (or even further uniform lighting) might be provided using a light diffusor placed between the light source(s) and the second end 112, preferably close to or adjacent to the second end 112. Such an embodiment is e.g. shown in FIG. 5.

In some embodiments, the material of the main housing 105, the central housing 101 (and thereby the second end 112), the cover 102 (and thereby the first end 111) may e.g. be the same (transparent) material (see e.g. also FIGS. 13-15).

The embodiments of a cell culture chamber device 100 as illustrated in FIGS. 11 and 12 provides a very compact (in particular in a lengthwise direction) self-contained and fully functioning cell culture chamber device 100 or bioreactor where the gas exchanger (and if included, the humidifier) is arranged away from a central axis and/or an axis of rotation. In addition, the cell culture chamber device 100 has a petri-dish like design enabling easy and familiar handling.

FIG. 13 schematically illustrates a perspective view of a main housing 105 of a cell culture chamber device as disclosed herein.

Illustrated is a main housing 105 according to some embodiments and e.g. as shown in connection with the embodiments of FIGS. 11A-11E and 12 more clearly showing certain features and aspects.

The main housing 105 comprises a cavity or open space and a first port 104 providing access to the enclosure (the port 104 to align in an assembled state of the cell culture chamber device with a further port; see e.g. 150 in FIG. 14B) where the port 104 is in fluidic connection with the enclosure 110.

The main housing 105 furthermore comprises a central opening 185 to be aligned with or receiving the second end (see e.g. 112 in FIGS. 11A-11E and elsewhere) in the assembled state of the cell culture chamber device. The main housing 105 is, as mentioned, configured to compactly receive a central housing (see e.g. 101 in FIGS. 14A and 14B).

In the particular shown embodiment, the main housing 105 furthermore comprises a number (here four as an example) of wall structure elements or similar 145 each for holding and/or supporting a water, liquid, or moisturizing element for embodiments also comprising a humidifier (please see further in the following). In alternative embodiments, the wall structure elements or similar 145 may be omitted. The wall structure elements or similar 145 may be distributed more or less evenly in the cavity or open space of the main housing 105 about a central axis/a rotational axis of the cell culture chamber device to distribute weigh of the water, liquid, or moisturizing elements more evenly. The wall structure elements or similar 145 may also provide structural integrity and/or support the received central housing.

In some embodiments and as shown, each (or some) wall structure element(s) or similar 145 comprises a cut-out or passage 141 forcing gas or air flow into close(r) proximity to a gas permeable membrane (see e.g. 120 in FIGS. 10, 11A, and 12).

Contained air or gas is in connection with a grid-like structure of the central housing (see e.g. 130 and 101 in FIGS. 14A and 14B and elsewhere) and finally the gas permeable membrane (see e.g. 120 in FIGS. 10, 11A, and 12). The grid-like structure provides support to the membrane while still allowing gas or air coming in contact with the membrane for gas exchange. The membrane may be secured, e.g. by welding, press-fitting, or gluing, to the grid-like structure. The gas permeable membrane is as mentioned configured to exchange gases, e.g. oxygen and carbon dioxide, with the content of the enclosure.

This readily provides a circumferential gas exchanger not blocking any line of sight between the first end and the second end in part defining the enclosure.

The function of the circumferential gas exchange system and the circumferential humidifier is further explained further in connection with FIG. 10.

FIGS. 14A and 14B schematically illustrate two perspective views of a central housing of a cell culture chamber device as disclosed herein.

Illustrated in FIGS. 14A and 14B is a first or central housing 101, in a transparent material, as disclosed herein more clearly illustrating the grid-like structure 130. Further illustrated are the second end 112, one or more fiducial and/or identification markers 155, one or more markings 115 in the form of a number of concentric circles 115 as an example, the gas exchange intake and outlet in the form of a double vent or similar 140, the further port 150, an aligning element 131.

FIG. 15 schematically illustrates a perspective view of a cover of a cell culture chamber device as disclosed herein.

Illustrated in FIG. 15 is a cover 102 in a transparent material. Further illustrated are the end 111, the closable and/or sealable port (first) port 103, and a number of level or fill-rate indicators 190.

In alternative embodiments, the diffusor 175 is not an optical diffusor but a diffusor 175 with respect to another type of illumination or visualisation signal, e.g. an acoustic diffusor or a diffusor for electromagnetic radiation different than light.

In some other alternative embodiments, the diffusor 175 (as disclosed herein) is replaced by a suitable reflector, e.g. a parabolic reflector, e.g. for use with front-lighting embodiments (or front-application of another type of illumination or visualisation signal) either in addition to or as an alternative to back-lighting or back-emission of another illumination or visualisation signal.

FIG. 16 schematically illustrates communication between a plurality of incubators and a user interface device.

Schematically illustrated is an incubator 200 as disclosed herein according to some embodiments. The incubator 200 comprises at least one registration and/or detection device 220 that e.g. or preferably is integrated with the incubator 200 as disclosed herein.

The at least one registration and/or detection device 220 may e.g. be an imaging or vision system or device and e.g. comprise or be one or more cameras configured to obtaining still images and/or video of the content of the enclosure as disclosed herein.

The illustrated incubator 200 further comprises one or more processing units 802 connected via one or more communications and/or data buses 801 to an electronic memory and/or electronic storage 803, one or more signal transmitter and receiver communications elements 804 (e.g. one or more selected from the group comprising cellular, Bluetooth, WiFi, etc. communications elements) for communicating via a computer network, the Internet, and/or the like 809, one or more optional (e.g. graphical and/or physical) user interface elements 807 e.g. including an optional display, and the one or more registration and/or detection devices/cameras 220.

In some embodiments, the incubator 200 comprises a number of cameras 220 equal to the number of cell culture chamber devices (see e.g. 100 elsewhere) that the incubator 200 is configured to received where each camera 220 is configured to capture videos and images of a particular cell culture chamber device or more particularly of an enclosure (see e.g. 110 elsewhere) of such device. Accordingly, it is possible to provide a video feed or a number of pictures e.g. together with additional information of individual respective cell culture chamber devices/enclosures, i.e. enabling online monitoring of such.

In some embodiments and as illustrated, the incubator 200 is configured to communicate via the network 809 with at least one external computational device, e.g. with one or more additional incubators 200′ that may or may not be located at the same physical location as the incubator 200. In at least some embodiments, the one or more additional incubators 200′ (or at least one or some thereof) correspond(s) to the incubator 200.

Captured videos and/or pictures may be stored locally and/or elsewhere, e.g. in a cloud storage connected to the network 809.

In some embodiments, a user interface device 250 is connected, at least at some times, with the incubator 200 via the network 809 and/or another network (such as a local network). The user interface device 250 may e.g. be a suitably programmed computational device, e.g. like a PC, laptop, computer, server, client, smart-phone, tablet, etc.

The user interface device 250 may e.g. be configured for online monitoring of the incubator 200. In some embodiments, the user interface device 250 is configured to display in a user interface on a screen a video online feed or a latest single or series of pictures for each incubator as obtained by the incubator 200 via its cameras 220. Additional data, such as current rotation speed, rotational direction, ID, etc. for each particular cell culture chamber device may also be obtained and transmitted to the user interface device 250 e.g. to be displayed on the device together with the video or image(s) for a respective cell culture chamber device. In addition to data that is specific to the cell culture chamber device(s), data for the incubator 200 may also be obtained and provided e.g. one or more of current temperature, current pH value, current humidity, current CO₂, O₂, and/or N₂ level(s), etc. of the incubation chamber (see e.g. 201 elsewhere) of the humidifier 200 as obtainable by a number of appropriate sensors located appropriately in the humidifier 200. The humidifier 200 may e.g. also send an alert or alarm to the user interface device 250 if certain one or more parameters is/are outside an acceptable range of values, above or below an accepted value, etc. (e.g. if the measured current temperature exceeds a given temperature threshold or value, etc.).

The online feed or the pictures may readily enable manual inspection of the state of contained spheroids, e.g. their size, their orbit, etc., which might prompt a user to want to change, e.g. increase, the rotational speed, if for example the spheroids now have become larger and thereby heavier (prompting for an increased rotational speed).

The ID of a particular cell culture chamber device may e.g. be obtained automatically by capturing an image or video of one or more fiducial and/or identification markers or codes (see e.g. 155, 180 elsewhere) and performing appropriate image analysis. In a similar manner, the presence of bubbles and/or an actual volume of cell culture media contained in a specific enclosure of a cell culture chamber device 100 may also be obtained and presented by capturing an image or video of a number of suitable level or fill-rate indicators (see e.g. 190 elsewhere).

In some embodiments, the user interface device 250 is further configured to perform data logging and/or documentation e.g. collecting and storing data such as temperature, humidity level, rotational speed, e.g. over time and e.g. including averages as well as duration and number of pauses (without rotation), etc. for at least some, e.g. all, of the cell culture chamber devices. This may e.g. be supplemented with video(s) and/or still image(s). The data of the data logging or documentation may e.g. be stored (e.g. also) in a cloud computing environment.

The user interface device 250 is at least in some embodiments configured to obtain (and present) data from a number of incubator 200, 200′.

In addition to obtaining data and information, the user interface device 250 may comprise input elements of its user interface for obtaining selections and other input from a user. This may e.g. be a new rotational speed setting for a (one or more) particular cell culture chamber device 100. It may also be start and stop commands for the cell culture chamber devices on an individual level.

Also illustrated as connected to the network 809 is an optional client computer or device 255. This may have the same functionality (or a super- or sub-set thereof) as described for the user interface device 250. The user may e.g. log-in on either 250 or 255 to be able to receive information and data and, if supported, provide commands, cause changes, etc. The client 255 may also be used instead of a user interface device. The client computer or device 250 may e.g. be a suitably programmed computational device, e.g. like a PC, laptop, computer, server, client, smart-phone, tablet, etc. Instead of or in addition to a client computer or device 255 and/or one or more additional incubators 200′, the one or more processing units 802 of the incubator 200 is/are configured to communicate via the network 809 with at least one other external computational device, e.g. a server computer or device, a network connected storage device, etc.

Any of the mentioned types of external computational device that the incubator 200 may communicate with may e.g. be a cloud computing (and/or storage) device.

In some embodiments, one of a group of incubators 200, 200′ is configured to be a ‘master’ while the rest is configured to be respective ‘slaves’ in relation to organising communication, exchange of information, etc. with the user interface device 250 (and/or the client 255) where the master communication with the user interface device 250 (and/or the client 255) and distribute data to the slave units. Additionally, the master also collects information from the slave units and pass it on to the user interface device 250 (and/or the client 255). Alternatively, another communications setup than master/slave may be used, e.g. a peer-to-peer setup, one to many setup, etc.

In some alternative embodiments, the at least one registration and/or detection device 220 is not an imaging or vision system or device configured to obtaining still images and/or video of the content of the enclosure as disclosed herein. In such alternative embodiments, the at least one registration and/or detection device 220 may e.g. be configured for registration of sound or acoustic waves (e.g. ultrasound) or for registration of electromagnetic radiation different than light (e.g. infrared, x-rays) as disclosed herein.

Some preferred embodiments have been shown in the foregoing, but it should be stressed that the invention is not limited to these, but may be embodied in other ways within the subject matter defined in the following claims.

It should be emphasized that the term “comprises/comprising” when used in this specification is taken to specify the presence of stated features, elements, steps or components but does not preclude the presence or addition of one or more other features, elements, steps, components or groups thereof.

In the claims enumerating several features, some or all of these features may be embodied by one and the same element, component or item. The mere fact that certain measures are recited in mutually different dependent claims or described in different embodiments does not indicate that a combination of these measures cannot be used to advantage.

In the claims, any reference signs placed between parentheses shall not be constructed as limiting the claim. The word “comprising” does not exclude the presence of elements or steps other than those listed in a claim. The word “a” or “an” preceding an element does not exclude the presence of a plurality of such elements.

The mere fact that certain measures are recited in mutually different dependent claims does not indicate that a combination of these measures cannot be used to an advantage.

It will be apparent to a person skilled in the art that the various embodiments of the invention as disclosed and/or elements thereof can be combined without departing from the scope of the invention as defined in the claims.

1. An incubator configured to receive a predetermined number, at least one or a plurality, of cell culture chamber devices, each cell culture chamber device comprising an enclosure configured to contain a cell culture media, the incubator comprising
a housing comprising an incubation chamber configured to contain at least a respective part of the cell culture chamber devices when received by the incubator, and
at least one registration and/or detection device being integrated with the incubator and being configured to register and/or detect an illumination or visualisation signal after passing, reflecting, or propagating through at least a part of the enclosure of at least one of the predetermined number of cell culture chamber devices when received by the incubator.

2. The incubator according to claim 1, wherein the incubator further comprises at least one rotational drive unit, each rotational drive unit
configured to, or releasably, receive at least one of the cell culture chamber devices, and
configured to rotate a cell culture chamber device, received by the rotational drive unit about a predetermined rotational axis of the received cell culture chamber device, the predetermined rotational axis being a predetermined central axis of the received cell culture chamber device or of the enclosure of the received cell culture chamber device.

3. The incubator according to claim 1, wherein the incubator comprises an openable and closable door or lid and the incubation chamber comprises at least one incubation chamber wall, the at least one incubation chamber wall and the door or lid, when closed, defining the incubation chamber at least in part, and wherein a first or an inner side of the door or lid comprises the at least one registration and/or detection device where the at least one registration and/or detection device is/are arranged facing so that an enclosure of at least one received cell culture chamber device is within a field of view of registration and/or detection of at least one of the at least one registration and/or detection device.

4. The incubator according to claim 3, wherein the incubator comprises the predetermined number of registration and/or detection devices, where each registration and/or detection device is arranged so that a central axis of a field of view of registration and/or detection of a respective registration and/or detection device at least substantially aligns with a central axis of a respective enclosure of a received cell culture chamber device.

5. The incubator according to claim 1, wherein the at least one registration and/or detection device is/are an imaging or vision system or device and the illumination or visualisation signal is ultraviolet, visible, infrared, and/or near-infrared light.

6. The incubator according to claim 5, wherein the incubator further comprises one or more light sources configured to illuminate at least a first end, or a part or window thereof, of an enclosure of one or more cell culture chamber devices received by the incubator, and where the one or more light sources is/are arranged in the door or lid facing one or more received cell culture chamber devices.

7. The incubator according to claim 2, wherein at least one respective drive unit of the at least one rotational drive unit comprises one or more light or illumination sources configured to illuminate at least a second end, or a part or window thereof, of an enclosure of a cell culture chamber device received by the respective drive unit.

8. The incubator according to claim 2, wherein at least one respective drive unit of the at least one rotational drive unit comprises a hollow rotational shaft comprising a light guide or other light or illumination element configured to illuminate at least a second end, or a part or window thereof, of an enclosure of a cell culture chamber device received by the respective drive unit.

9. The incubator according to claim 2, wherein a respective motor part of the at least one rotational drive unit is located outside the incubation chamber and inside the housing.

10. The incubator according to claim 1, wherein the incubator further comprises a fan or ventilation unit arranged in the incubation chamber and configured to cause an air flow inside the incubation chamber in response to a control signal, and wherein the fan or ventilation unit further comprises a UVC light configured to emit UVC light inside the incubation chamber.

11. The incubator according to claim 10, wherein at least a part of an inner surface of the incubation chamber, or at least a part of the at least one incubation chamber wall and/or a first or an inner side of the door or lid, comprises a UVC reflecting material or coating.

12. The incubator according to claim 1, wherein the incubator further comprises
one or more processing units,
an electronic memory and/or electronic storage, and
one or more signal transmitter and receiver communications elements configured to communicate with a network,
wherein the one or more processing units is/are configured to communicate via the network with at least one external computational device.

13. The incubator according to claim 1, wherein the incubator comprises a cell culture chamber device for the growing of cell cultures and tissues, the cell culture chamber device comprising
an enclosure configured to contain a cell culture media, and
a first end, a second end, and at least one connecting wall connecting the first and the second ends, where the first end, the second end, and the at least one connecting wall at least in part defines the enclosure,
wherein the first end, or a part or window thereof, is substantially transparent and the second end and/or at least one of the at least one connecting wall, or a respective part or window thereof, is/are substantially transparent or is/are substantially translucent, where the first end or the part or window thereof is configured to be optically or otherwise aligned, at least for some period of time or periodically, with the second end or the part or window thereof and/or with the at least one of the at least one connecting wall or the part or window thereof so that light or another illumination or visualisation signal, transmitted through or by the second end or the part or window thereof and/or through or by the connecting wall or the part or window thereof into the enclosure is transmitted or propagates through at least a part of the cell culture media and out through the first end or the part or window thereof to outside the enclosure.

14. An incubator system comprising
a first incubator according to claim 1,
at least a second incubator according to claim 1, and
a user interface device and/or a client computer or device,
wherein
the first incubator is configured as a master unit and the at least a second incubator is configured as a slave unit,
the master unit is configured to control communication and/or data exchange between the master unit and all slave units with the user interface device and/or the client computer or device,
the user interface device and/or the client computer or device is configured to obtain user input control data and communicate the user input control data to the master unit, and
the master unit is configured to change or adapt operation in response to at least a part of the received user input control data and/or communicating at least a part of the received user input control data to at least one slave unit, the at least one slave unit configured to change or adapt operation in response to at least a part received user input control data.

15. An incubator configured to receive a predetermined number, at least one or a plurality, of cell culture chamber devices, each cell culture chamber device comprising an enclosure configured to contain a cell culture media, the incubator comprising
a housing comprising an incubation chamber configured to contain at least a respective part of the cell culture chamber devices when received by the incubator, and
an UVC arrangement configured to expose an interior the incubation chamber directly with UVC light, the UVC arrangement comprising one or more UVC lights arranged inside the incubation chamber.

16. The incubator according to claim 15, wherein the one or more UVC lights is/are one or more rotating UVC lights configured to rotate about a predetermined axis within the incubation chamber thereby sweeping the interior of the incubation chamber.

17. An incubator configured to receive a predetermined number, at least one or a plurality, of cell culture chamber devices, each cell culture chamber device comprising an enclosure configured to contain a cell culture media, the incubator comprising
a housing comprising an incubation chamber configured to contain at least a respective part of the cell culture chamber devices when received by the incubator, wherein the incubation chamber comprises a circular cross-section substantially perpendicular to a first or length wise direction.

18. An incubator configured to receive a predetermined number, at least one or a plurality, of cell culture chamber devices each cell culture chamber device comprising an enclosure configured to contain a cell culture media, the incubator comprising
a housing comprising an incubation chamber configured to contain at least a respective part of the cell culture chamber devices when received by the incubator, and
at least one rotational drive unit, each rotational drive unit
configured to, releasably, receive at least one of the cell culture chamber devices, and
configured to rotate a cell culture chamber device, received by the rotational drive unit, about a predetermined rotational axis of the received cell culture chamber device, the predetermined rotational axis of the received cell culture chamber device or of the enclosure of the received cell culture chamber device,
wherein a respective motor part of the at least one rotational drive unit is located outside the incubation chamber and inside the housing.

19. An incubator configured to receive a predetermined number, at least one or a plurality, of cell culture chamber devices each cell culture chamber device comprising an enclosure configured to contain a cell culture media, the incubator comprising
a housing comprising an incubation chamber configured to contain at least a respective part of the cell culture chamber devices when received by the incubator, and
a fan or ventilation unit arranged in the incubation chamber and configured to cause an air flow inside the incubation chamber in response to a control signal.
